(19) United States
(12) Patent Application Publication
(10) Pub. No.: US 2025/0213373 A1
(43) Pub. Date: Jul. 3, 2025

(54) SPINAL IMPLANT SYSTEM, IMPLANT INSERTER ASSEMBLY, IMPLANTATION METHODS AND KIT

(71) Applicant: Spinal Surgical Strategies, Inc., a Nevada Corporation D/B/A Kleiner Device Labs, Incline Village, NV (US)

(72) Inventors: Alan Burkholder, Denver, CO (US); Gregory Causey, Erie, CO (US); Jeffrey Kleiner, Denver, CO (US)

(73) Assignee: Spinal Surgical Strategies, Inc., a Nevada Corporation D/B/A Kleiner Device Labs, Incline Village, NV (US)

(21) Appl. No.: 18/701,317

(22) PCT Filed: Oct. 25, 2022
(86) PCT No.: PCT/US2022/047774

§ 371 Date: Apr. 15, 2024

Related U.S. Application Data
(60) Provisional application No. 63/271,281, filed on Oct. 25, 2021.

(51) Int. Cl. A61F 2/44; A61F 2/30; A61F 2/46

(52) U.S. Cl. CPC A61F 2/447; A61F 2/30771; A61F 2/4601; A61F 2/4611; A61F 2002/3092; A61F 2002/3093; A61F 2002/4619; A61F 2002/4631; A61F 2002/4681

(57) ABSTRACT

An interbody spinal implant/fusion cage and implant inserter system for facilitating interbody fusion in the lumbar spine using bone graft while reducing the number of passes made through the patient's disc space and the sensitive surrounding tissue by integrating bone graft delivery into the inserter system. The system combines insertion of the interbody implant with bone graft delivery directly into the disc space. The system includes the interbody implant, an interbody cover plate, an implant inserter assembly, a cover plate inserter, a bone graft plunger and a bone graft funnel. The inserter assembly includes a large rectangular cannula which attaches to a large rectangular posterior opening in the interbody implant instead of a round cannula. The rectangular cannula approximates the shape of the annulotomy and provides a larger cross-sectional area for passage of a much larger quantity of bone graft material without jamming. Related methods and kits are also disclosed.

### RELATED APPLICATION

This application claims priority to U.S. Provisional Patent Application No. 63/271,281, filed Oct. 25, 2021, the disclosure of which is incorporated herein by reference in its entirety.

### FIELD OF THE INVENTION

Embodiments of the invention relate generally to surgical implants and instruments, methods and kits relating to same, and more particularly, to surgical implants, instruments, methods and kits for use in spinal procedures involving interbody spinal fusion.

### BACKGROUND OF THE INVENTION

Transforaminal and posterior interbody fusion of the spine are used to treat degenerative disc disease (DDD) and up to Grade 1 spondylolisthesis of the lumbar spine at one level or at least two contiguous levels. DDD is defined as back pain of discogenic origin with degeneration of the disc confirmed by history and radiographic studies. Known techniques involve multiple sequential steps of inserting a graft funnel, using a spatula to adjust and position the graft in the disc space so that an interbody can be inserted, placing the interbody and then following with additional graft introduction. Such techniques require multiple passes into the patient's disc space and surrounding tissue, which involves greater risk of injury, such as nerve trauma.

It is therefore desirable to provide improved surgical instruments that system is designed to reduce the number of passes, which translates into a reduction in nerve trauma. and thereby provide superior patient outcomes and utility for surgeons.

### SUMMARY OF THE INVENTION

The invention includes a surgical system comprising an interbody implant, an implant inserter assembly and means for releasably connecting the implant inserter assembly to the interbody implant. The interbody implant includes an open body having top and bottom (i.e., superior and inferior) surfaces, opposed side surfaces, and posterior and anterior surfaces, an open center and a central strut extending longitudinally through the open center, from the anterior surface towards the posterior surface. The posterior surface includes a posterior opening extending along at least 30% of the posterior surface between the side surfaces. The side surfaces each include a side opening 40a, 40b extending along at least 50% of the side surfaces. The side openings and posterior opening allow for bone graft to flow through the posterior surface of the interbody implant and through the side surfaces and into a patient's surrounding disc space. The implant inserter assembly includes a graft cannula lock tube, an inserter tube configured to telescopically receive the graft cannula lock tube therein, a release tube configured to telescopically receive the inserter tube therein, an impactor configured to insertably engage the graft cannula lock tube, inserter tube and release tube, and means for releasably interconnecting the graft cannula lock tube, inserter tube and release tube, wherein the graft cannula lock tube, inserter tube and release tube cooperate to form a serial cannula system, and have uniform internal areas to improve transmission of bone graft therethrough and into the posterior opening of the interbody implant.

The invention further includes a surgical system comprising an interbody implant, an implant inserter assembly, means for releasably connecting the implant inserter assembly to the interbody implant, an interbody cover plate and a cover plate inserter. The interbody implant includes an open body having top and bottom (i.e., superior and inferior) surfaces, opposed side surfaces, and posterior and anterior surfaces, an open center and a central strut extending longitudinally through the open center, from the anterior surface towards the posterior surface. The posterior surface includes a posterior opening extending along at least 30% of the posterior surface between the side surfaces, and the side surfaces each include a side opening extending along at least 50% of the side surfaces. The side openings and posterior opening allow for bone graft to flow through the posterior surface of the interbody implant and through the side surfaces and into a patient's surrounding disc space. The implant inserter assembly includes a graft cannula lock tube, an inserter tube configured to telescopically receive the graft cannula lock tube therein, a release tube configured to telescopically receive the inserter tube therein, an impactor configured to insertably engage the graft cannula lock tube, inserter tube and release tube, and means for releasably interconnecting the graft cannula lock tube, inserter tube and release tube, wherein the graft cannula lock tube, inserter tube and release tube cooperate to form a serial cannula system, and have uniform internal areas to improve transmission of bone graft therethrough and into the posterior opening of the interbody implant. The interbody cover plate is configured to removably engage the posterior opening of the interbody implant. The cover plate includes a cover plate body and a cover plate cam. The cover plate body includes a posterior surface defining a threaded opening configured for engagement with the cover plate inserter and two integrally-formed arms extending from the cover plate body on opposed sides of the threaded opening and configured to engage the side surfaces of the interbody implant and lock the cover plate to the interbody implant. The cam includes a hexalobe feature for engagement with the cover plate inserter.

Another embodiment of the invention includes a kit for spinal surgery and comprises an interbody implant, an implant inserter assembly, an interbody cover plate configured to removably engage the posterior opening of the interbody implant, a cover plate inserter configured for engagement with the cover plate, a disassembly pliers configured to disconnect the graft cannula lock tube, inserter tube and release tube from one another. a graft funnel configured to removably engage the distal end of the graft cannula lock tube, a graft plunger configured to insertably engage an opening of the graft funnel and to advance bone graft from the graft funnel through the graft cannula lock tube, through the interbody implant and into the disc space, and means for removing or repositioning the interbody implant. The interbody implant includes an open body having top and bottom (i.e., superior and inferior) surfaces, opposed side surfaces, and posterior and anterior surfaces, an open center and a central strut extending longitudinally through the open center from the anterior surface towards the posterior surface. The posterior surface includes a posterior opening extending along at least 30% of the posterior surface between the side surfaces. The side surfaces each include a side opening extending along at least 50% of the side surfaces. The side openings and posterior opening allow for bone graft to flow through the posterior surface of the interbody implant and through the side surfaces and into a patient's surrounding disc space. The implant inserter assembly is releasably connectable to the interbody implant and includes a graft cannula lock tube, an inserter tube configured to telescopically receive the graft cannula lock tube therein, a release tube configured to telescopically receive the inserter tube therein, and an impactor configured to insertably engage the insertably engage the graft cannula lock tube, inserter tube and release tube. The graft cannula lock tube, inserter tube and release tube cooperate to form a serial cannula system, and have uniform internal areas to improve transmission of bone graft therethrough and into the posterior opening of the interbody implant.

The details of one or more examples of the invention are set forth in the description below. Other features, objects, and advantages of the invention will be apparent from the detailed description of the examples and also from the drawing and the appending claims.

### BRIEF DESCRIPTION OF THE DRAWINGS

Embodiments of the invention are further described but are in no way limited by the following illustrations.

FIG. 1 is a top plan view of a spinal implant and inserter system and kit according to an embodiment of the present invention;

FIG. 2A is a top perspective view of an interbody implant and cover plate of the system of FIG. 1;

FIG. 2B is a top perspective view of the cover plate shown in FIG. 2A;

FIG. 2C is another top perspective view of the cover plate shown in FIG. 2A, shown at a 90° angle to the view of FIG. 2B;

FIG. 3A is a bottom perspective view of a graft cannula lock tube of the system of FIG. 1;

FIG. 3B is an exploded bottom perspective view of an inserter tube of the system of FIG. 1, shown with the interbody implant of FIG. 2A;

FIG. 3C is a bottom perspective view of a release tube of the system of FIG. 1;

FIG. 3D is a bottom perspective view of an impactor of the system of FIG. 1;

FIG. 4A is a top plan view of the assembled release tube and inserter tube of the system of FIG. 1;

FIG. 4B is a top plan view of the assembled release tube and inserter tube of the system of FIG. 1, shown at a 90° angle to the view of FIG. 4A;

FIG. 4C is a detailed partial side elevational view of the release tube and interbody implant of the system of FIG. 1;

FIG. 4D is a detailed partial side elevational view of the release tube and inserter tube of the system of FIG. 1;

FIG. 4E is a detailed partial side elevational view of the release tube and inserter tube of the system of FIG. 1;

FIG. 4F is a detailed partial top perspective view of the release tube and inserter tube of the system of FIG. 1;

FIG. 5A is a side elevational view of the assembled release tube, inserter tube and interbody implant of the system of FIG. 1;

FIG. 5B is a side elevational view of the assembled graft cannula lock tube, release tube and inserter tube to form the implant inserter assembly of the system of FIG. 1, with interbody implant of the system of FIG. 1;

FIG. 5C is a detailed partial side perspective view of the release tube and interbody implant of the system of FIG. 1;

FIG. 5D is a detailed partial top plan view of the assembled graft cannula lock tube, release tube and inserter tube of the system of FIG. 1;

FIG. 6A is a top plan view of the assembled impactor, graft cannula lock tube, release tube, inserter tube and interbody implant of the system of FIG. 1;

FIG. 6B is a detailed partial top plan view of the release tube, impactor and interbody implant of the system of FIG. 1;

FIG. 6C is a detailed partial top perspective view of the release tube and interbody implant of the system of FIG. 1;

FIG. 6D is a detailed partial top plan view of the assembled impactor, graft cannula lock tube, release tube and inserter tube of the system of FIG. 1;

FIG. 6E is a detailed partial top plan view of the assembled impactor, graft cannula lock tube, release tube and inserter tube of the system of FIG. 1, shown at a 90° angle to the view of FIG. 6D;

FIG. 7A is a top perspective view of a first step of separating the graft cannula lock tube from other components of the implant inserter assembly of the system of FIG. 1;

FIG. 7B is a top perspective view of a second step of separating the graft cannula lock tube from other components of the implant inserter assembly of the system of FIG. 1;

FIG. 7C is a top perspective view of a third step of separating the graft cannula lock tube from other components of the implant inserter assembly of the system of FIG. 1;

FIG. 7D is a top perspective view of a fourth step of separating the graft cannula lock tube from other components of the implant inserter assembly of the system of FIG. 1;

FIG. 8A is a top perspective view of the implant inserter assembly of the system of FIG. 1, with the graft funnel of the system of FIG. 1;

FIG. 8B is a top perspective view of the graft plunger of the system of FIG. 1;

FIG. 8C is a top perspective view of the implant inserter assembly, graft funnel and graft plunger of the system of FIG. 1;

FIG. 9A is a top perspective view of the interbody implant and implant inserter assembly of the system of FIG. 1, with a syringe containing bone graft attached thereto in an alternate embodiment of the invention;

FIG. 9B is a top perspective view of the interbody implant and implant inserter assembly of the system of FIG. 1, with the syringe of FIG. 9A shown in a deployed state to deliver the bone graft therein to the implant inserter assembly and interbody implant;

FIG. 10A is an exploded view of the interbody implant, cover plate, implant inserter assembly components and a cover plate inserter of the system of FIG. 1;

FIG. 10B is a detailed partial top perspective view of the cover plate and cover plate inserter of FIG. 10A, with the cover plate shown in an unlocked position;

FIG. 10C is a detailed partial top perspective view of the cover plate and cover plate inserter of FIG. 10A, with the cover plate shown in a locked position;

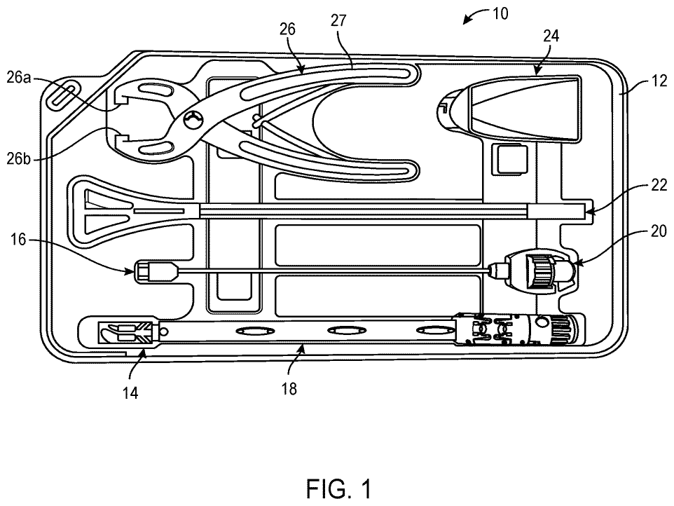

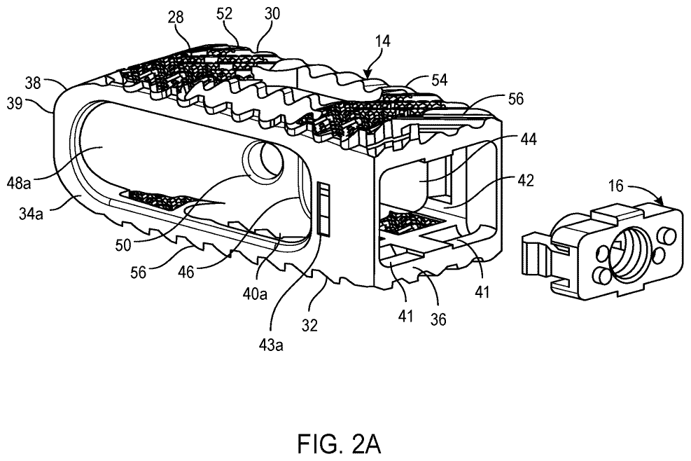

FIG. 1 1A is a top perspective view of the interbody implant, implant inserter assembly components and cover plate inserter of the system of FIG. 1;

FIG. 1 1B is a detailed partial bottom perspective view of the interbody implant and release tube of the system of FIG. 1;

FIG. 11C is a top plan view of the interbody implant, implant inserter assembly components and cover plate inserter of the system of FIG. 1;

FIG. 1 1D is another detailed partial bottom perspective view of the interbody implant and release tube of the system of FIG. 1;

FIG. 12A is a top perspective view of a first step of releasing the interbody implant from the implant inserter assembly of the system of FIG. 1 using the release tube;

FIG. 12B is a detailed partial top perspective view of the step shown in FIG. 12A;

FIG. 12C is a top perspective view of a second step of releasing the interbody implant from the implant inserter assembly of the system of FIG. 1;

FIG. 12D is a detailed partial top perspective view of the step shown in FIG. 12C;

FIG. 13 is a detailed partial top perspective view of the interbody implant and release tube of the system of FIG. I;

FIG. 14A is an exploded top perspective view of a slap hammer and inserter slap hammer adaptor according to an embodiment of the invention, along with the implant inserter assembly and interbody implant of the system of FIG. I;

FIG. 14B is a top perspective view of the slap hammer, inserter slap hammer adaptor, implant inserter assembly and interbody implant shown in FIG. 14A, as assembled together;

FIG. 15A is an exploded top plan view of a side grip remover/repositioner instrument according to an embodiment of the invention, along with the implant inserter assembly and interbody implant of the system of FIG. I;

FIG. 15B is a top plan view of the side grip remover/repositioner instrument, implant inserter assembly and interbody implant shown in FIG. 15A, as assembled together;

FIG. 1 5C is a detailed partial top plan view of the side gnp remover/repositioner instrument of FIGS. 15A-15B in a first, unthreaded position;

FIG. 15D is a detailed partial top plan view of the side grip remover/repositioner instrument of FIGS. 15A-15B in a second, threaded position;

FIG. 15E is a detailed partial bottom perspective view of the side grip remover/repositioner instrument, implant inserter assembly and interbody implant shown in FIG. 15A, when the side grip remover/repositioner instrument is in the first position of FIG. 15C;

FIG. 1 5F is another detailed partial bottom perspective view of the side grip remover/repositioner instrument, implant inserter assembly and interbody implant shown in FIG. 15A, when the side grip remover/repositioner instrument is in the second position of FIG. 15D;

FIG. 16A is a top plan view of a hooked remover/repositioner instrument according to an embodiment of the invention, along with the interbody implant of the system of FIG. 1;

FIG. 16B is a detailed partial top perspective view of the hooked remover/repositioner instrument and interbody implant shown in FIG. 16A;

FIG. 1 6C is a detailed partial top perspective view of the hooked remover/repositioner instrument and interbody implant shown in FIGS. 16A and 16B, with the hooked remover/repositioner instrument shown connected to the interbody implant;

FIG. 17A is a top plan view of a threaded/cover plate remover/repositioner instrument according to an embodiment of the invention, along with the interbody implant of the system of FIG. 1;

FIG. 17B is a detailed partial top perspective view of the threaded remover/repositioner instrument and interbody implant shown in FIG. 17A;

FIG. 1 7C is a detailed partial top perspective view of the threaded remover/repositioner instrument and interbody implant shown in FIGS. 17A and 17B, with the threaded remover/repositioner instrument shown connected to the interbody implant;

FIG. 18 is a top perspective environmental view of the implant inserter assembly and interbody implant of the system of FIG. 1 in a patient's disc space;

FIG. 19A is a top perspective environmental view of the implant inserter assembly and interbody implant of the system of FIG. 1, showing the removal of the impactor thereof;

FIG. 19B is a top perspective environmental view of the implant inserter assembly and interbody implant of the system of FIG. 1, as seen with the impactor removed from the implant inserter assembly thereof;

FIG. 20 is a top perspective environmental view of the implant inserter assembly, interbody implant and graft funnel of the system of FIG. 1;

FIG. 21 is a top perspective environmental view of the implant inserter assembly, interbody implant, graft funnel and graft plunger of the system of FIG. 1;

FIG. 22A is a top perspective environmental view of the implant inserter assembly and interbody implant of the system of FIG. 1, showing the removal of the graft cannula lock tube thereof;

FIG. 22B is a top perspective environmental view of the implant inserter assembly and interbody implant of the system of FIG. 1, as seen with the graft cannula lock tube removed from the implant inserter assembly thereof;

FIG. 23A is an exploded top perspective environmental view of the implant inserter assembly and interbody implant of the system of FIG. 1, along with the cover plate and cover plate inserter;

FIG. 23B is a top perspective environmental view of the implant inserter assembly, interbody implant and cover plate inserter of the system of FIG. 1, as assembled, with the cover plate inserter in a first position;

FIG. 23C is another top perspective environmental view of the implant inserter assembly, interbody implant and cover plate inserter of the system of FIG. 1, as assembled, with the cover plate inserter in a second position;

FIG. 23D is a top perspective environmental view of the implant inserter assembly and interbody implant of the system of FIG. 1, showing the removal of the cover plate inserter;

FIG. 24A is a top perspective environmental view of the implant inserter assembly and interbody implant of the system of FIG. 1, showing a separation step for same;

FIG. 24B is a top perspective environmental view of the implant inserter assembly and interbody implant of the system of FIG. 1, showing the removal of the implant inserter assembly components;

FIG. 24C is a top perspective environmental view of the interbody implant of the system of FIG. 1, as implanted within the disc space;

FIG. 25 is a top perspective environmental view of the inserter slap hammer adaptor, implant inserter assembly and interbody implant;

FIG. 26A is a top perspective environmental view of the side gnp remover/repositioner instrument, implant inserter assembly and interbody implant, showing the removal of the interbody implant from the disc space;

FIG. 26B is a top perspective environmental view of the side grip remover/repositioner instrument and interbody implant, showing the removal of the interbody implant from the disc space;

FIG. 27A is a top perspective environmental view of the hooked remover/repositioner instrument, implant inserter assembly and interbody implant, showing the removal of the interbody implant from the disc space;

FIG. 27B is a top perspective environmental view of the hooked remover/repositioner instrument and interbody implant, showing the removal of the interbody implant from the disc space;

FIG. 28A is a top perspective environmental view of the threaded remover/repositioner instrument, implant inserter assembly and interbody implant, showing the removal of the interbody implant from the disc space; and

FIG. 28B is a top perspective environmental view of the threaded remover/repositioner instrument and interbody implant, showing the removal of the interbody implant from the disc space.

### DETAILED DESCRIPTION OF THE INVENTION

This invention includes embodiments of an interbody (i.e., intervertebral) spinal implant and inserter system for same. While the implant is used to facilitate interbody fusion in the lumbar spine from L2 to SI using autogenous and/or allogenic bone graft comprised of cancellous and/or corticocancellous bone graft to facilitate fusion, it is also envisioned to be adapted for use in other areas of the spine, and even other areas of the body, and further envisioned to be used with other graft materials. Thus, reference to “bone graft” or “bone graft material” should be readily understood to incorporate the possibility of other materials. The system is designed to reduce the number of passes made through the patient's disc space and surrounding tissue, which translates into a reduction in nerve trauma, by integrating bone graft delivery into the delivery system. Instead of the known technique of inserting a graft funnel, using a spatula to adjust and position the bone graft in the disc space so that an interbody implant can be inserted, placing the interbody implant and then following with additional bone graft introduction, this system inserts the integrated interbody implant/inserter assembly so that all of the graft delivery and distribution is accomplished through the cannula tube of the inserter assembly. The system further facilitates the application of bone graft when the disc space is in its final, distracted position, allowing the appropriate, non-diluted volume of applied bone graft. As further discussed below, the system also includes an inserter having a large rectangular cannula which attaches to a large rectangular posterior opening in the interbody implant instead of a round cannula. The rectangular cannula approximates the shape of the annulotomy and allows for a larger cross-sectional area to be used, which allows for the passage of a much larger quantity of bone graft material without jamming.

The system of the present invention 1s designed to allow minimal manipulation of the patient's tissues (including sensitive nerve structures) when performing a posterior interbody fusion by combining the insertion of the interbody implant (e.g., fusion cage) with a simultaneous application of bone graft material (also referred to herein as ‘bone graft’) into the fusion platform and directly into the disc space. The lack of lateral walls of the interbody implant allows a broad spectrum of graft consistencies and materials to flow into the prepared disc space while the disc area is in its distracted position.

An embodiment of the implant and inserter system IO and its components is shown in FIGS. 1-1 7C and further described below. The system IO includes a tray 12 that is configured to contain the other components therein as a kit and may include a cover or lid (not shown) to secure the other components therein. The system 10 further includes an interbody implant 14, an interbody cover plate 16 configured to removably engage the interbody implant 14, an implant inserter assembly 18, a cover plate inserter 20, a bone graft plunger 22, a bone graft funnel 24, and disassembly pliers 26. In the embodiment as shown, the interbody implant 14 is preassembled with the inserter assembly 18 and the interbody cover plate 16 is preassembled with the cover plate inserter 20. Such preassembly is not required in alternate embodiments. Yet further, embodiments of the system 10 are envisioned wherein one or more of the items recited above are excluded from tray 12.

### Interbody Implant

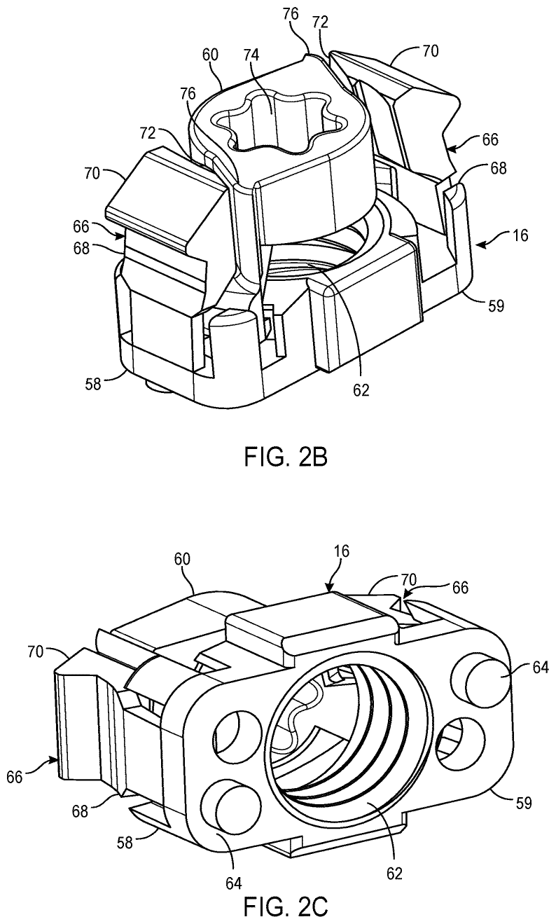

FIG. 2A shows the interbody implant 14 and cover plate 16. The interbody implant 14 (also shown in FIG. 13) has a ‘cage’-like configuration, and includes an open body 28 having top and bottom (i.e., superior and inferior) surfaces 30, 32, opposed side surfaces 34a, 34b (optionally being mirror images of each other), and posterior and anterior surfaces 36, 38. The anterior surface 38 includes a convexly-shaped, or bullet-shaped “nose” 39 that facilitates insertion of the interbody implant 14 into a patient's disc space. The side surfaces 34a, 34b each include a side opening 40a, 40b formed therein. The side openings 40a, 40b are relatively large, extending along a significant portion (i.e., 50% or greater) of the side surfaces 34a, 34b, respectively. This structure provides interbody implant 14 with minimal to no lateral walls, which minimizes impediments to bone graft flow from within the open body 28 of the implant 14 to the surrounding disc space.

The posterior surface 36 of the interbody implant 14 includes one or more undercuts 41 and a posterior opening 42 formed therein. The undercut(s) 41 is configured to secure the implant inserter assembly 18 (i.e., a portion thereof) to the interbody implant 14, as further described below. The posterior opening 42 is relatively large, extending along a significant portion (i.e., 30% or greater) of the posterior surface 36. The side openings 40a, 40b and posterior opening 42 allow for bone graft to flow through the posterior surface 36 of the interbody implant 14 and through the side surfaces 34a, 34b and into the surrounding disc space. The side surfaces 34a, 34b also each include a side cutout 43a, 43b formed therein proximate the posterior surface 36. The side cutouts 43a, 43b are configured to secure the cover plate 16 to the interbody implant 14, as further described below.

The interbody implant's open body 28 further includes an open center 44 with a central strut 46 extending longitudinally through the open center 44, from the anterior surface 38 towards the posterior surface 36. The central strut 46 provide the interbody implant 14 with an “I beam” configuration. The central strut 46 does not fully extend to the posterior surface 36 and leaves the posterior opening 42 unobstructed for bone graft to flow therethrough. The central strut 42 is positioned anteriorly enough such that the cross-sectional area of the posterior opening 42 is substantially maintained through both side openings 40a or 40b. Because of this maintained cross-sectional area, large granules of bone graft that can pass through the inserter tube 80 can also pass through either side opening 40a or 40b without obstruction.

The central strut 46 includes graft ramps 48a, 48b formed on the sides thereof. The central strut 46 and graft ramps 48a, 48b direct the flow of bone graft through the interbody implant 14 (i.e., the open body 28). The ramps 48a, 48b have a gradual curvature that facilitates easier bone graft flow through the interbody implant 14 and into the surrounding disc space. In an embodiment, the ramps 48a, 48b are positioned to direct the bone graft to one side, e.g., as a uniportal design for PLIF purposes.

The central strut 46 also includes a center hole 50 formed therein (i.e., between graft ramps 48a, 48b). The center hole 50 is used for repositioning or removal of the interbody implant 14 with a generic hooked type instrument (such as a small curette or never hook) that is commonly available in the operative room, surgical suite, etc.

The top and bottom surfaces 30, 32 of the open implant body 28 each include one or more graft windows 52 that allow for direct contact between bone graft in the open body 28 and a patient's vertebral end plates adjacent the top and bottom surfaces 30, 32 (i.e., superior and inferior to the interbody implant 14). The top and bottom surfaces 30, 32 further include a lattice structure 54 configured to facilitate bone ingrowth from the adjacent vertebral end plates after implantation of the interbody implant 14. The top and bottom surfaces 30, 32 also include projections (e.g., teeth) 56 that engage the adjacent vertebrae and thereby restrict migration of the interbody implant 14 after its implantation.

In some embodiments, the interbody implant 14 is formed from implant grade titanium Ti6A14V alloy (ASTM F3001). Other biocompatible materials may also be used in alternate embodiments of the interbody implant. In other embodiments, the interbody implant 14 is 3D printed (i.e., additive manufacturing) from porous PEEK, ceramic or silicon nitride.

### Interbody Cover Plate

The cover plate 16 is configured to cover the posterior opening 42 of the interbody implant 14. The cover plate 16 is shown in detail in FIGS. 2B and 2C.

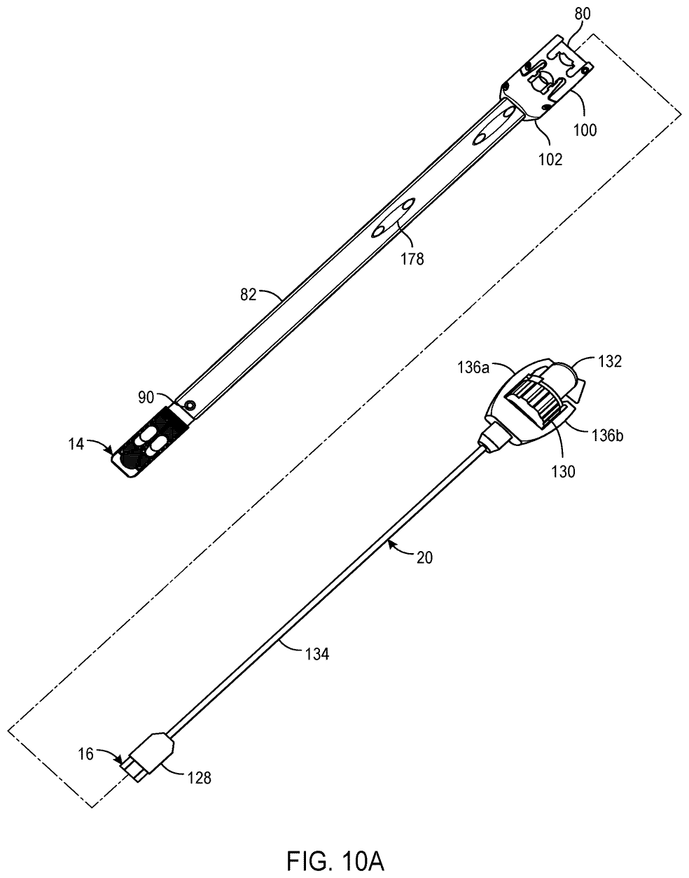

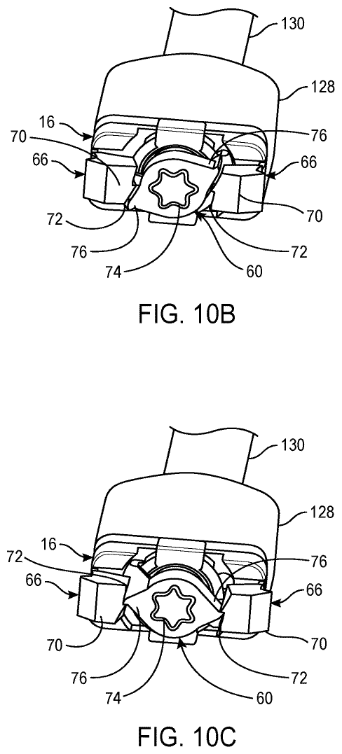

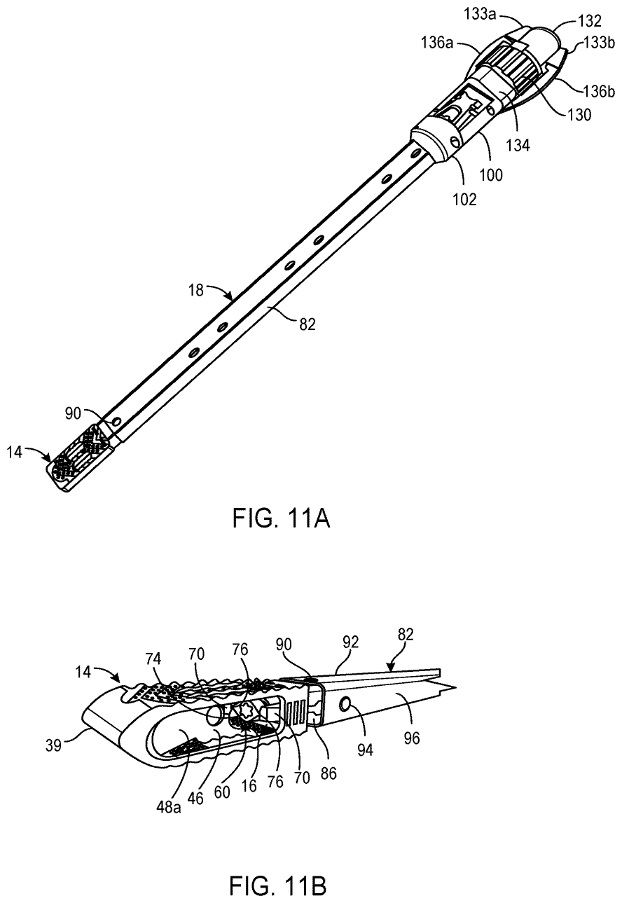

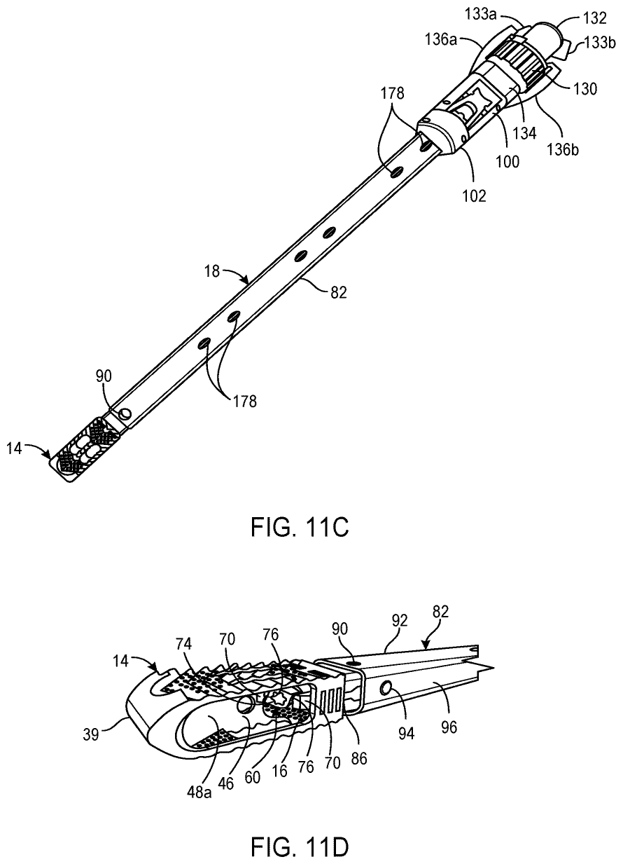

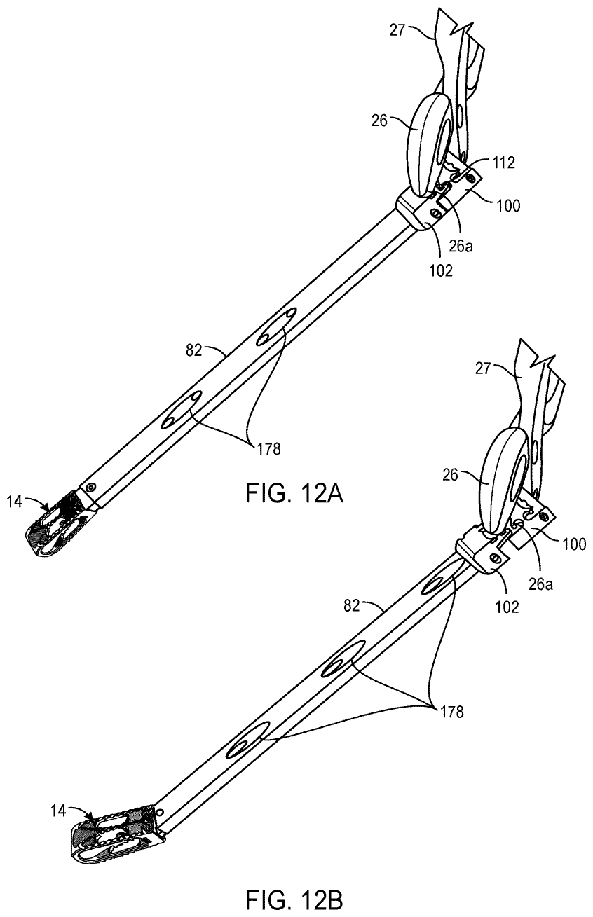

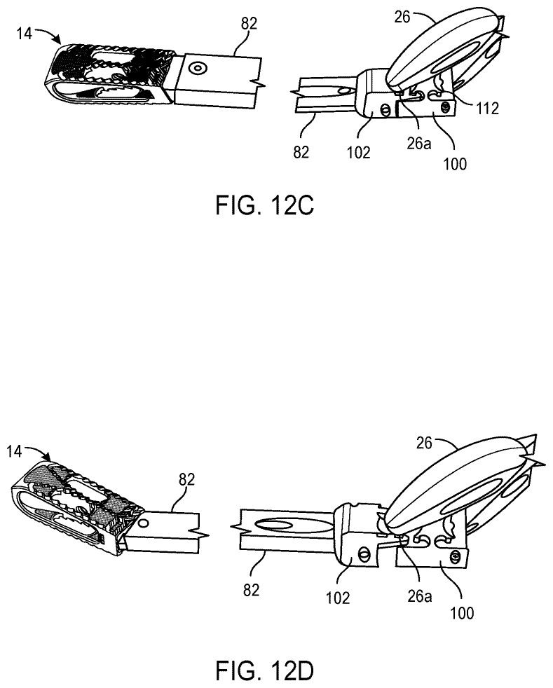

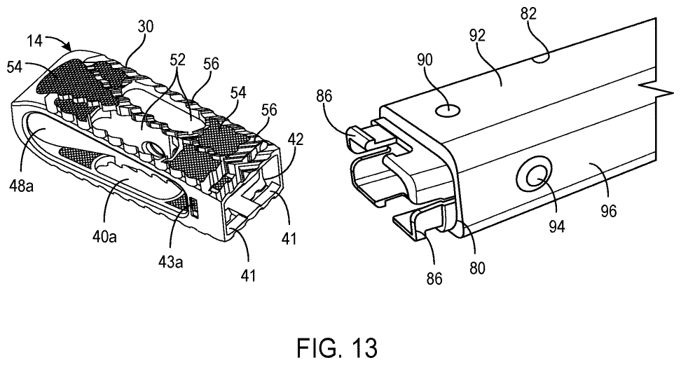

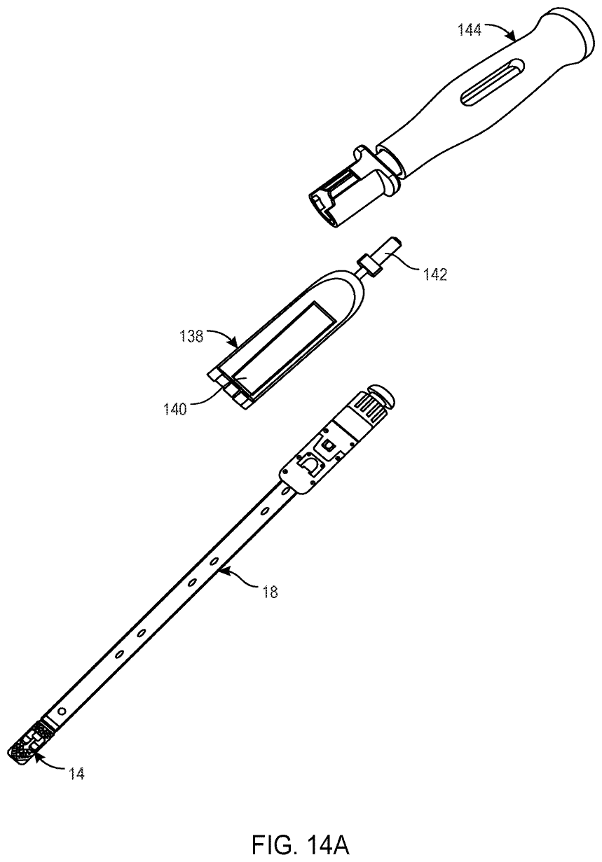

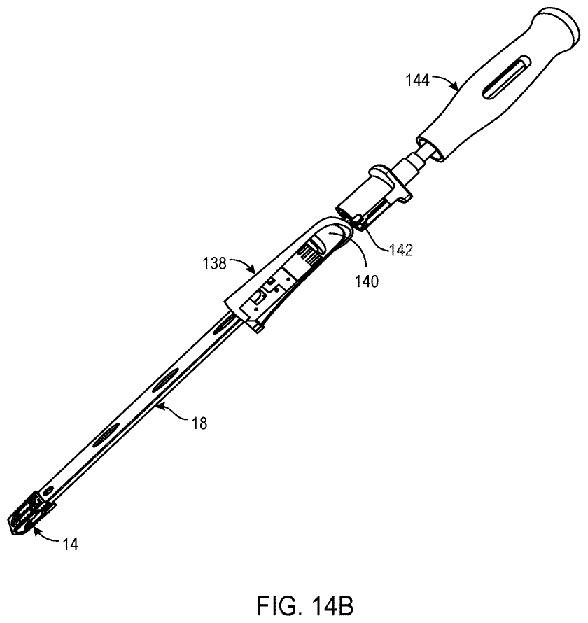

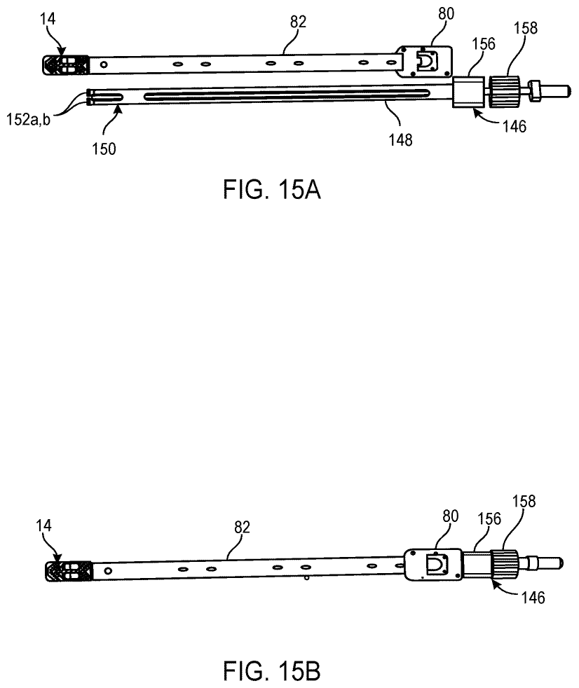

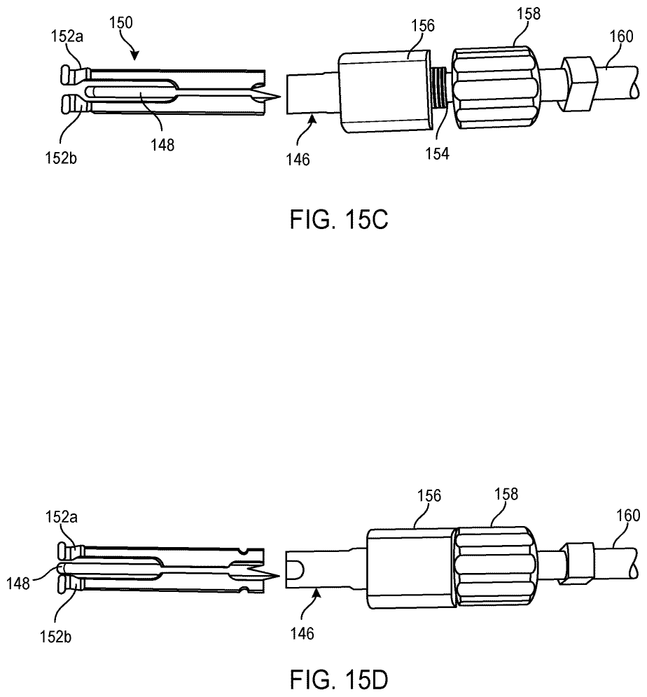

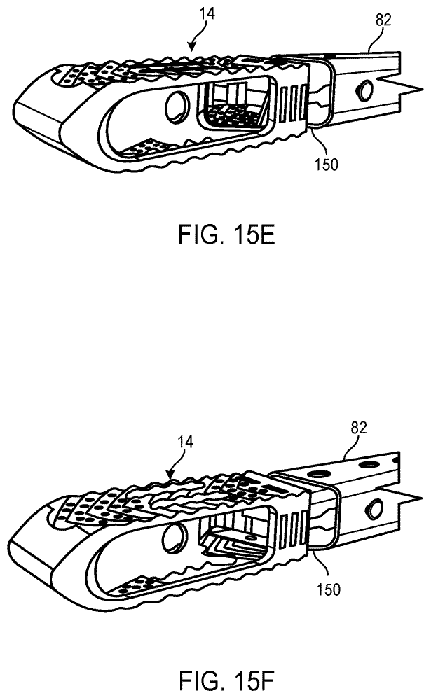

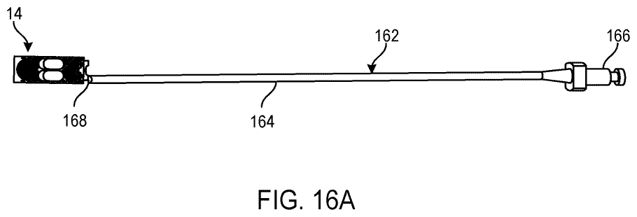

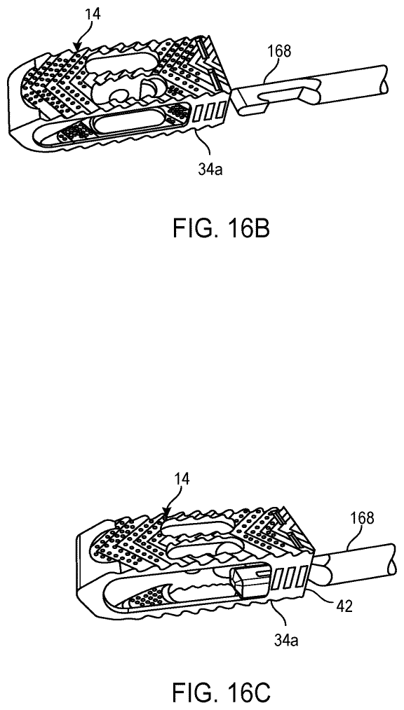

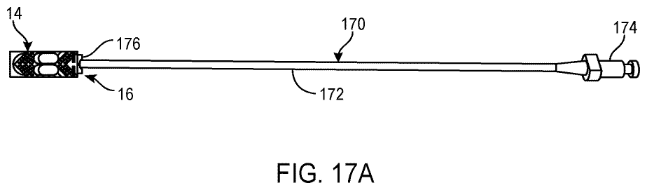

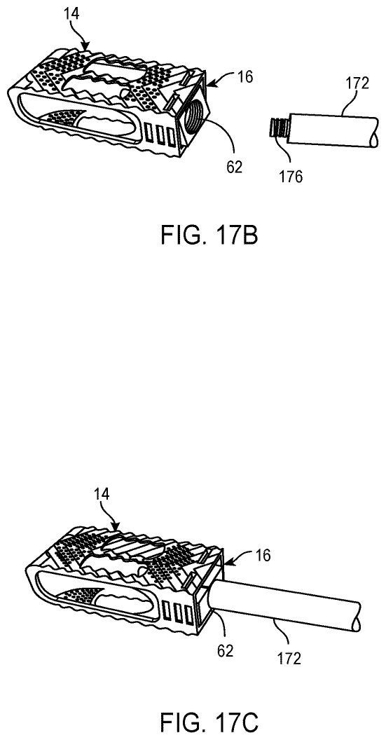

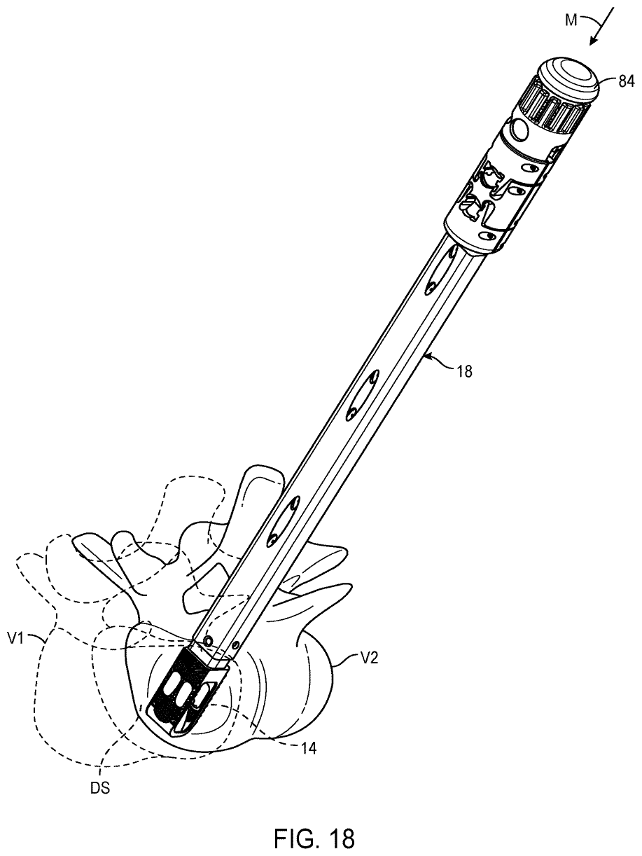

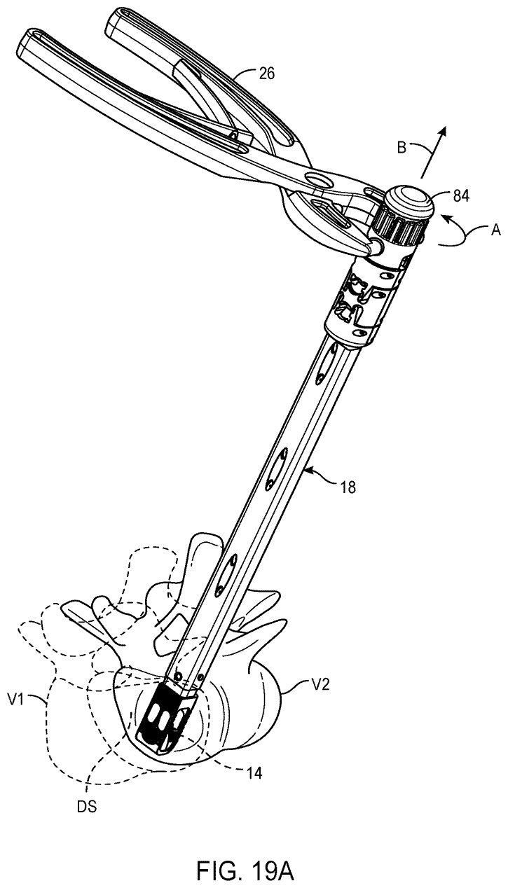

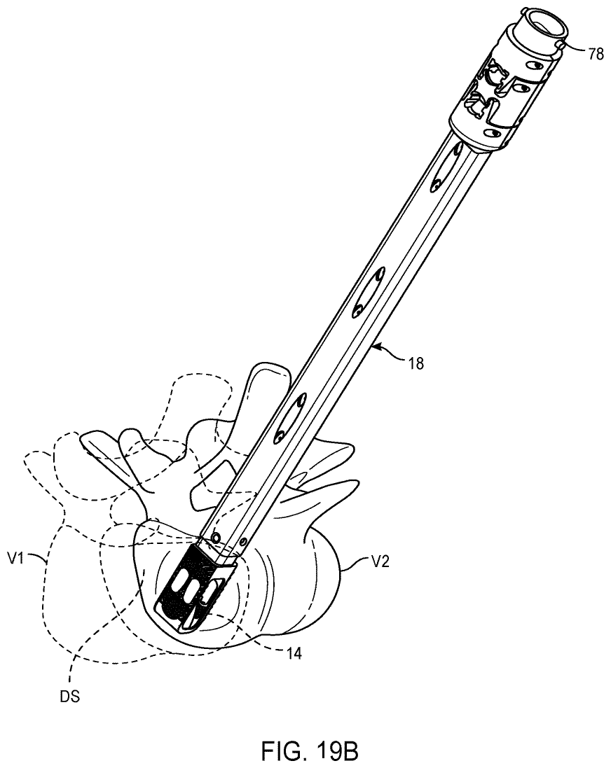

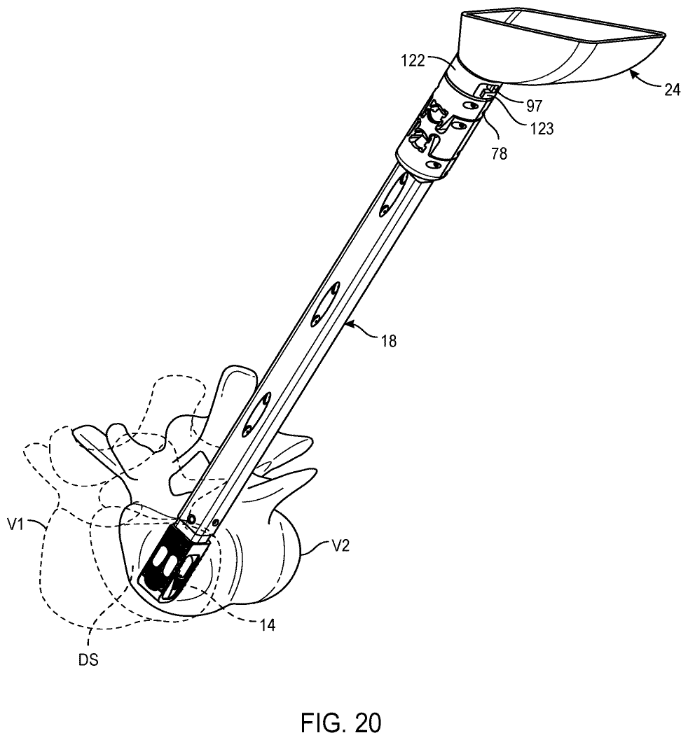

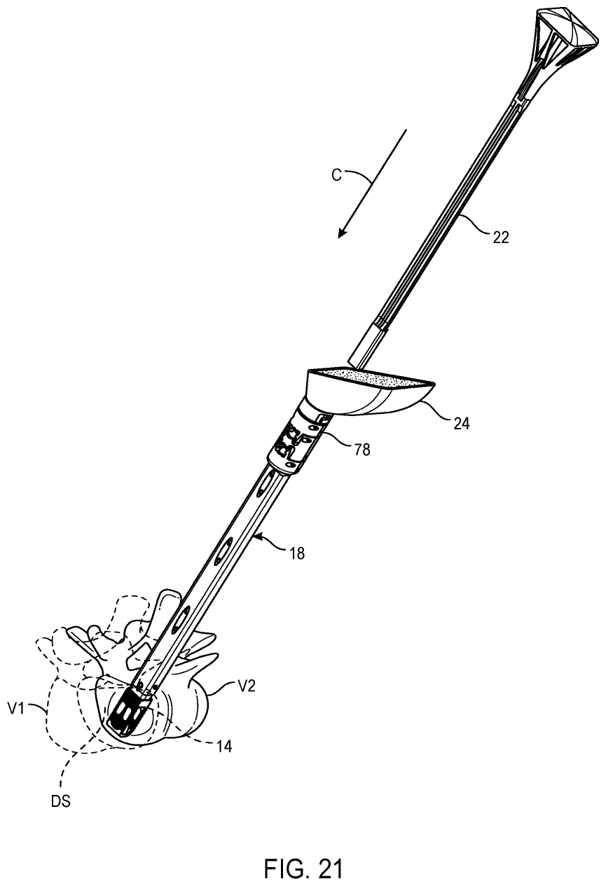

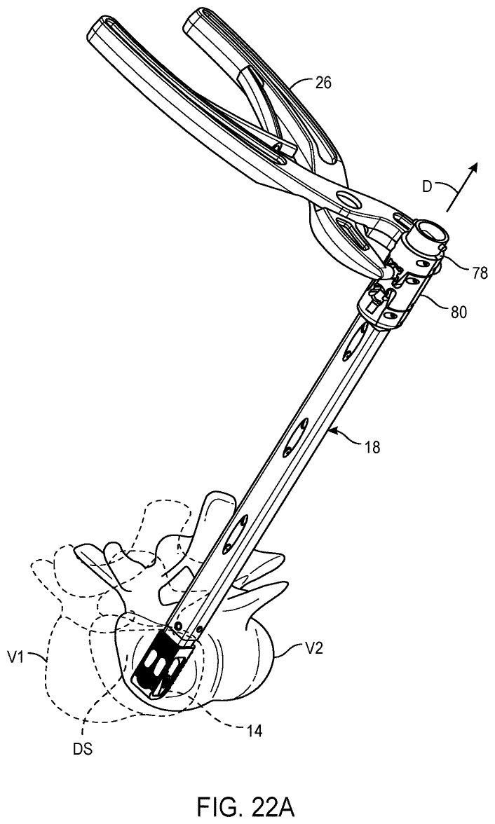

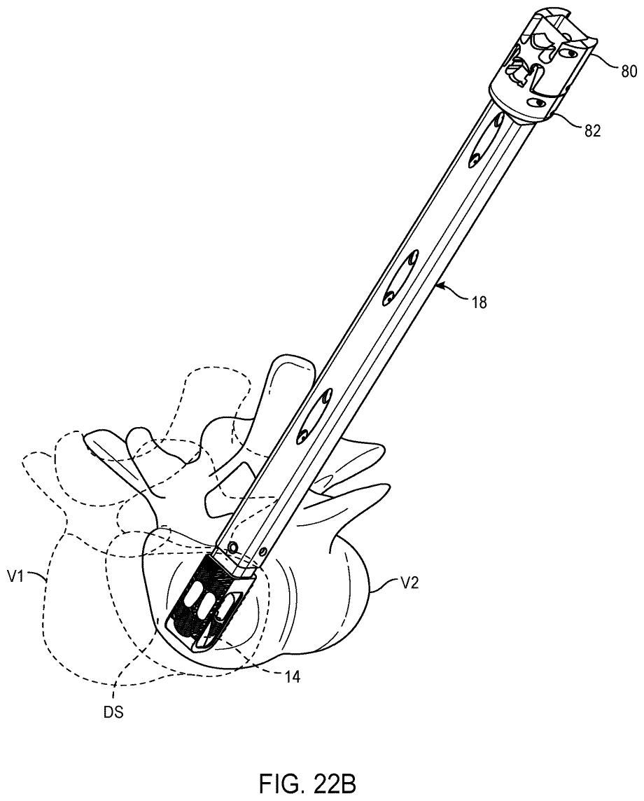

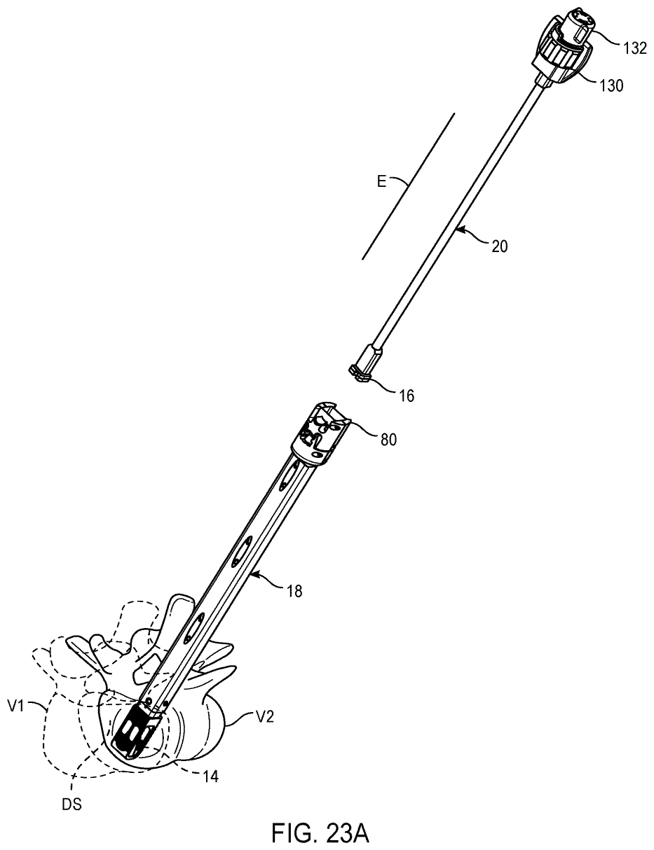

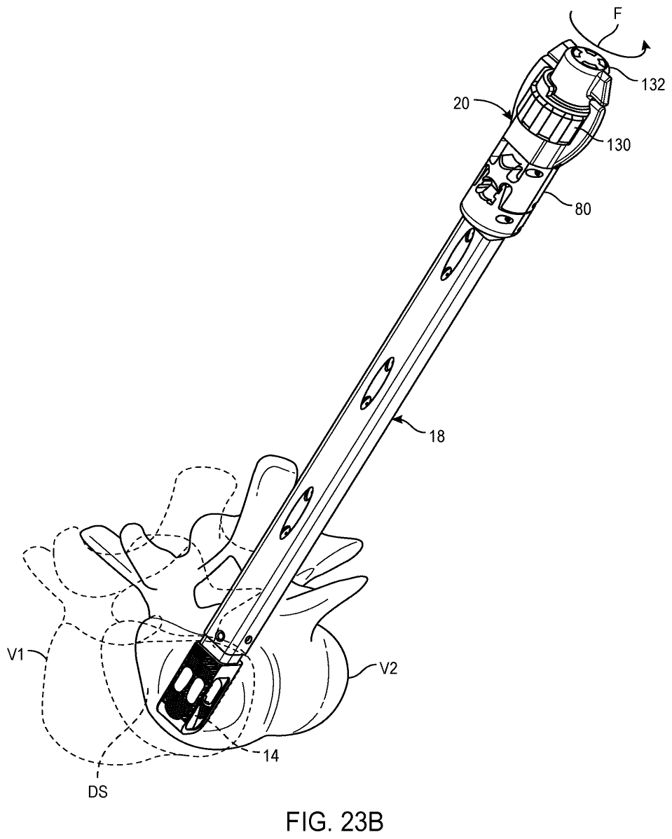

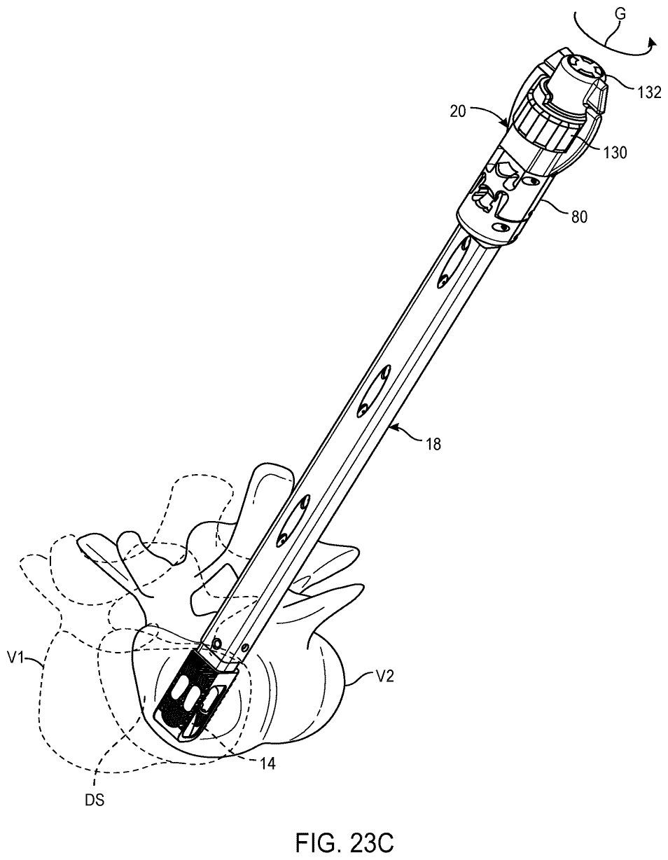

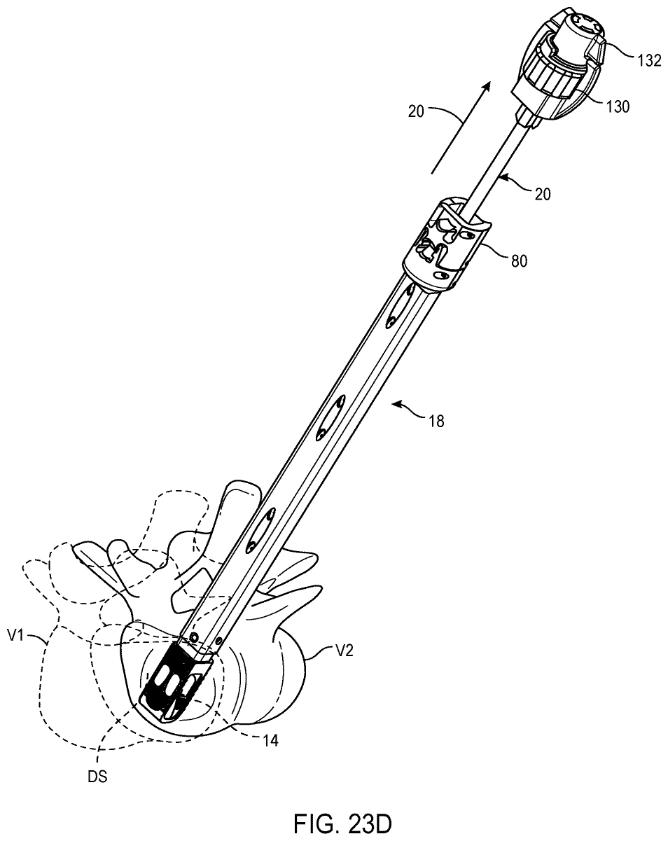

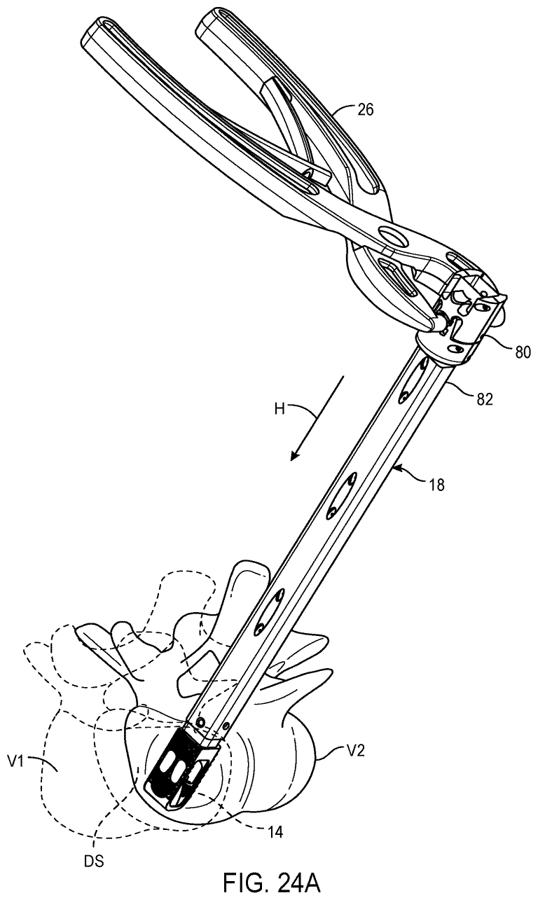

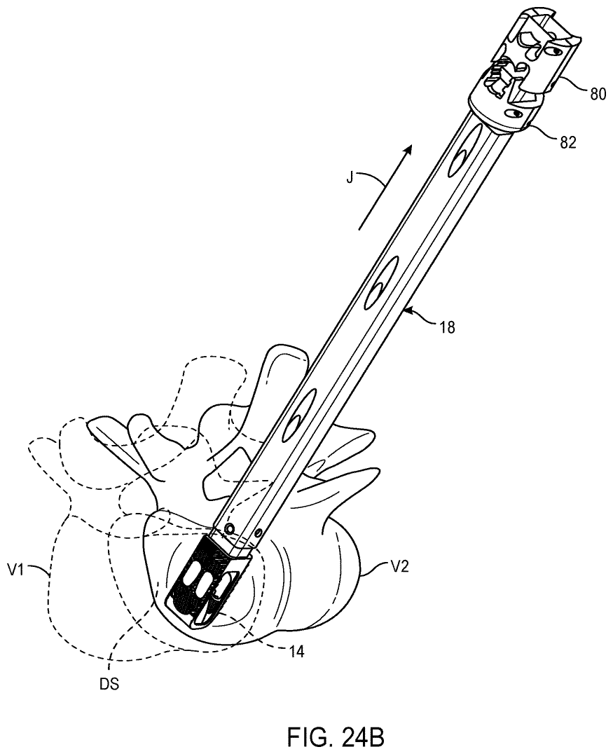

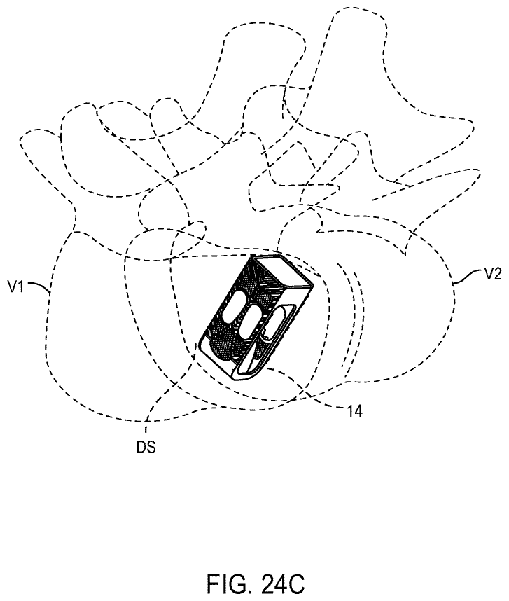

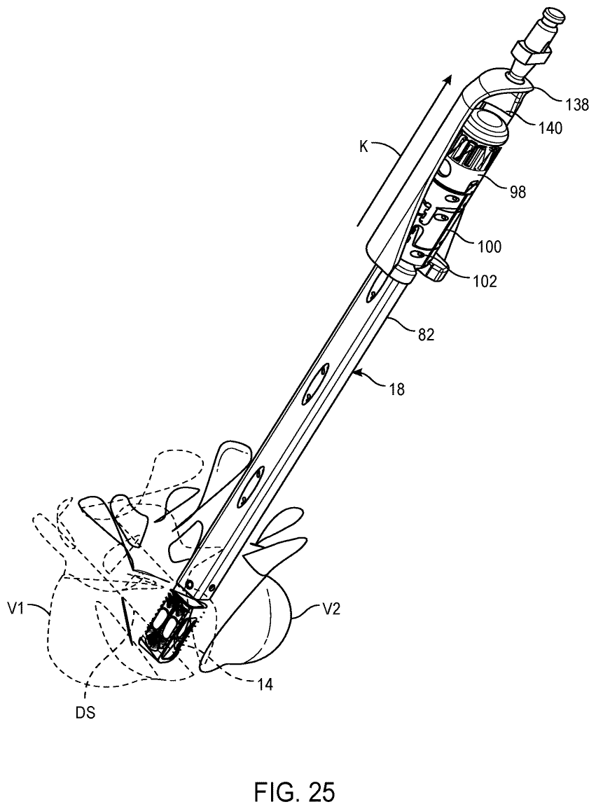

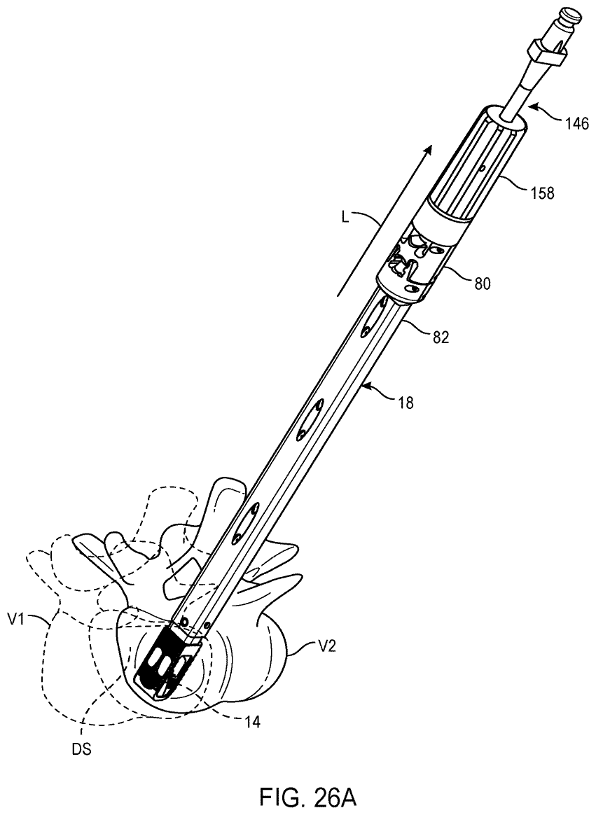

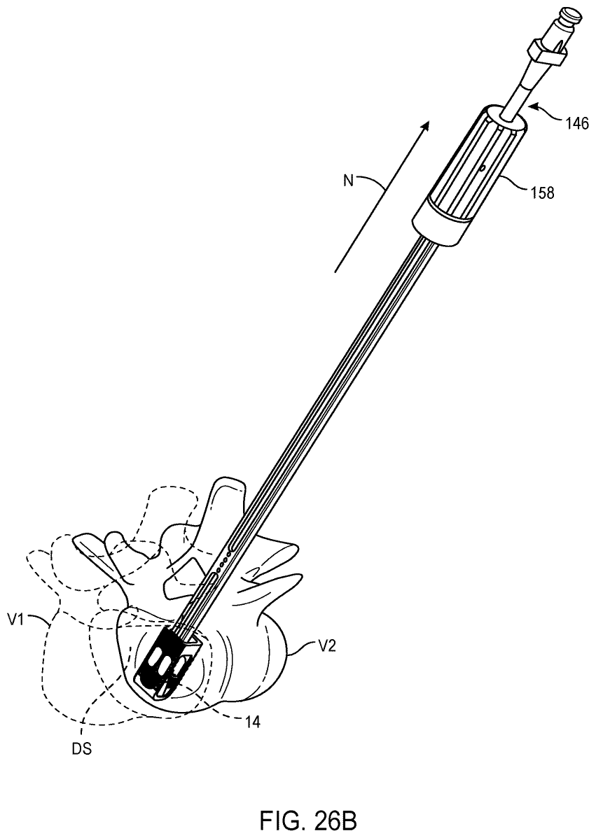

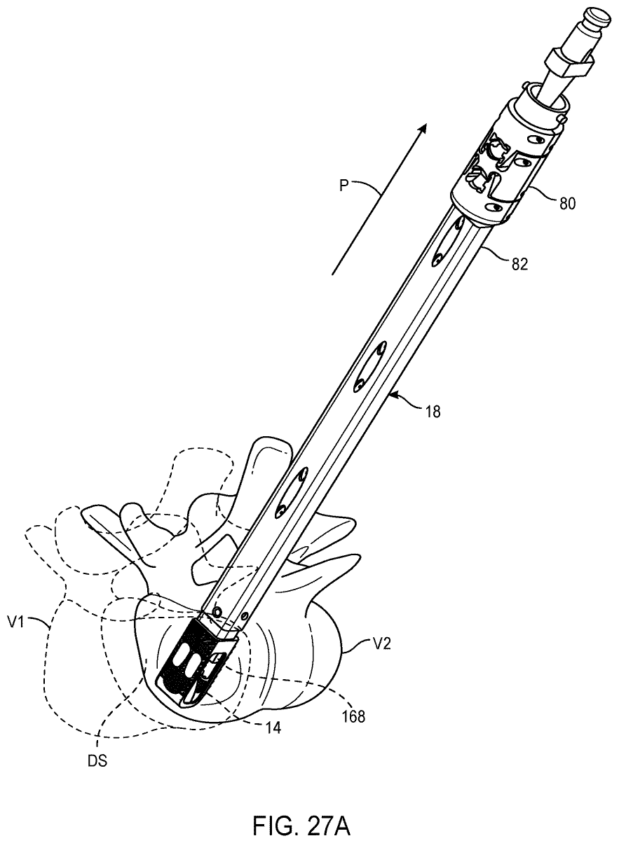

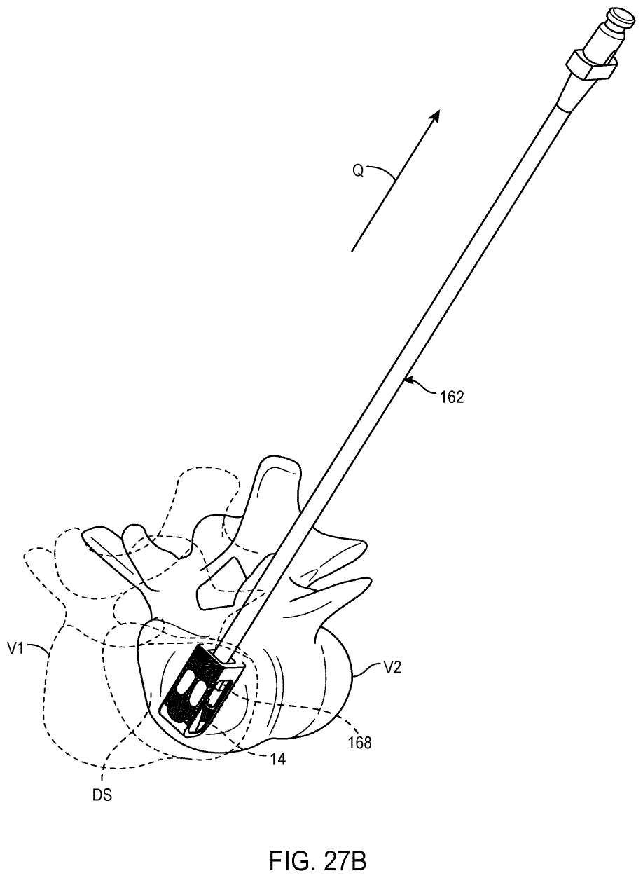

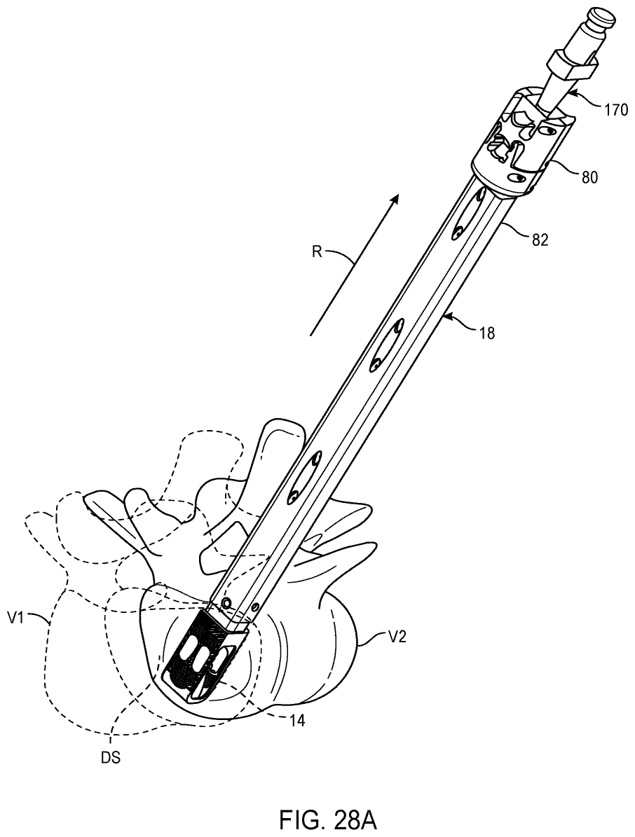

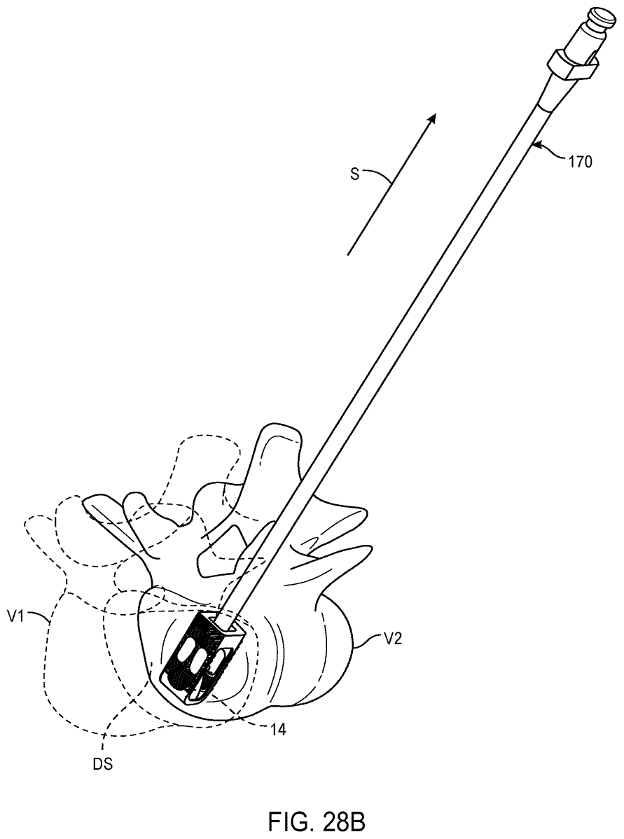

The cover plate 16 includes two components—a cover plate body 58 and a cover plate cam 60. The cover plate body 58 includes a posterior surface 59 defining a threaded opening, or interface, 62 therein that is configured for engagement with the cover plate inserter 20. The posterior surface 59 also includes a plurality of protrusions, or posts, 64 that are configured to insertably engage pockets, or apertures, on the cover plate inserter 20 in order to prevent rotation of the cover plate 16 during placement. Two integrally-formed arms 66 extend from the cover plate body 58 on opposed sides of the threaded opening 62 and each include a flex zone area 68, an outwardly-extending wedge-shaped end 70 and a vertically-extending tab, or ledge, 72 adjacent the wedge-shaped end 70. The arms 66 are configured to flex outwardly by bending at the flex zone area 68. The wedge-shaped ends 70 of the arms 66 are configured to insertably engage the side cutouts 43a, 43b of the interbody implant 14 and lock the cover plate 16 to the interbody implant 14, as described below and shown in FIGS. 10A-11D. The tabs 72 are configured to engage with the cam 60, as discussed below.

The cam 60 includes a hexalobe feature 74 for engagement with the cover plate inserter 20 and two locking members 76. The cam 60 can be rotated relative to the cover plate body 58 through a limited range of rotation of approximately 30°. The cam 60 engages the arms 66 during rotation and forces the arms 66 to flex outwardly by bending at the flex zone area 68. At the end of the cam's rotational range, the locking members 76 of the cam 60 engage with (i.e., snap past) the tabs 72 to prevent reverse rotation, which locks the arms 66 in the outward flexed orientation to prevent their movement.

In an embodiment, the cover plate 16 is 3D printed as a single piece. The cover plate 16 is formed to have sufficient attachment strength such that it can be used to extract the interbody implant 14 from the disc space in cases requiring removal and/or repositioning (as discussed below).

### Implant Inserter Assembly

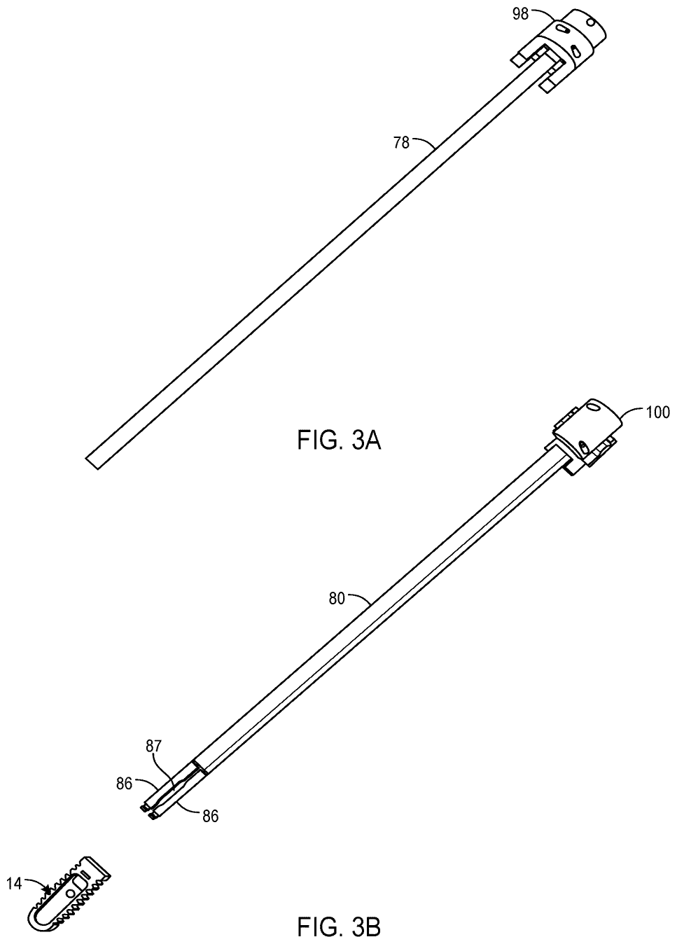

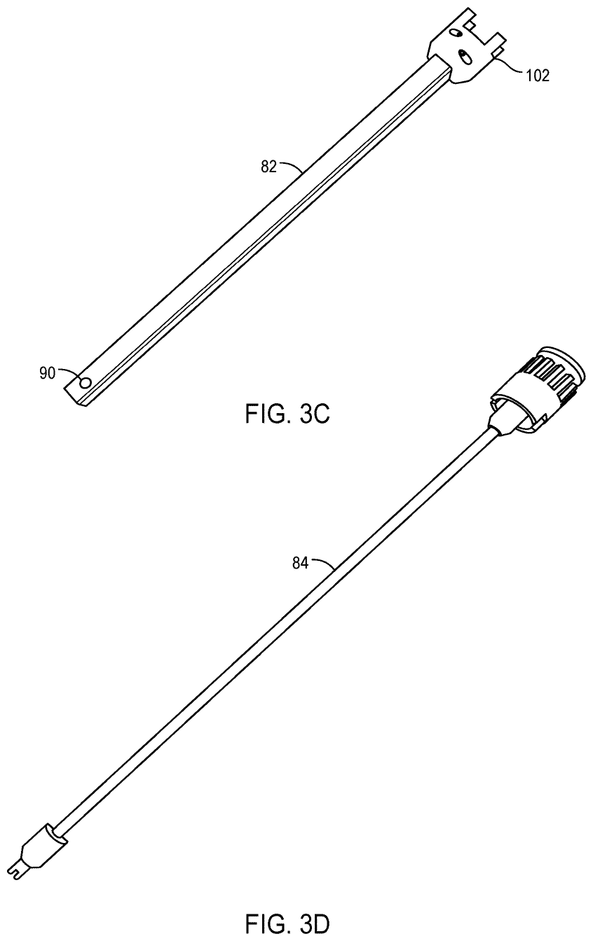

Embodiments of the components of the implant inserter assembly 18 are illustrated in FIGS. 3A-7D. The components constitute a series of telescoping tubes, or cannulas, that enable insertion of bone graft into and through the interbody implant 14 and surrounding area in the disc space of a patient's spine.

The inserter assembly 18 includes a graft cannula lock tube 78 as shown in FIG. 3A, an inserter tube 80 as shown in FIG. 3B (with the interbody implant 14), a release tube 82 as shown in FIG. 3C, and an impactor 84 as shown in FIG. 3D. All of these components cooperate to form a serial cannula system, as further discussed below. Further, all of the tubes 78, 80 and 82 have uniform internal areas to improve transmission of bone graft therethrough and into the posterior opening 42 of the interbody implant 14, with the size of the opening 42 maintained through both side surfaces 34a, 34b of the interbody implant 14, thereby avoiding a constrictive “wasp waist” impediment to bone graft flow into the implant 14 and ultimately to the disc space. The tubes of the inserter assembly 18 are secured to the interbody implant 14 in a robust fashion without compromising the internal cross-sectional area thereof. While the tubes are shown as having rectangular cross-sectional shapes, other shapes are also possible.

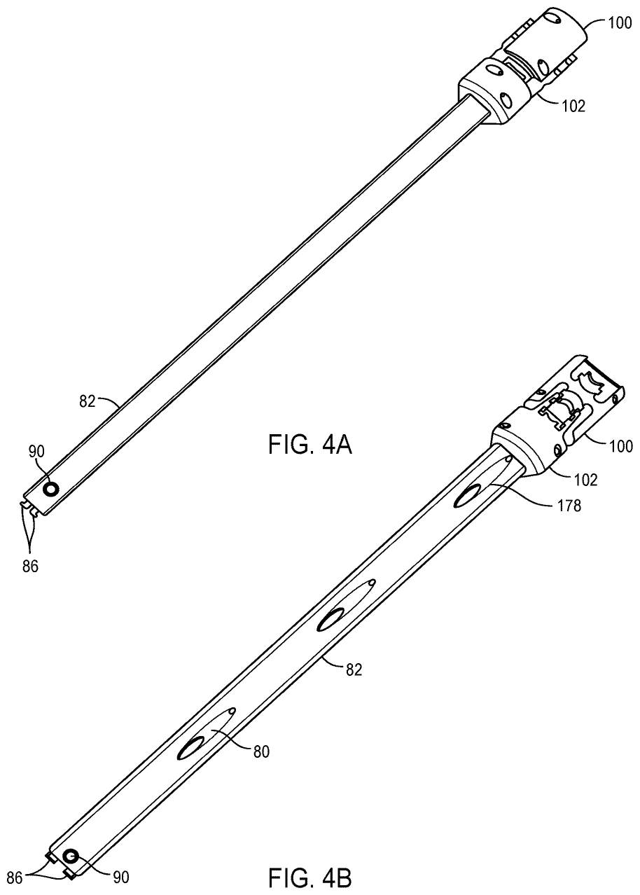

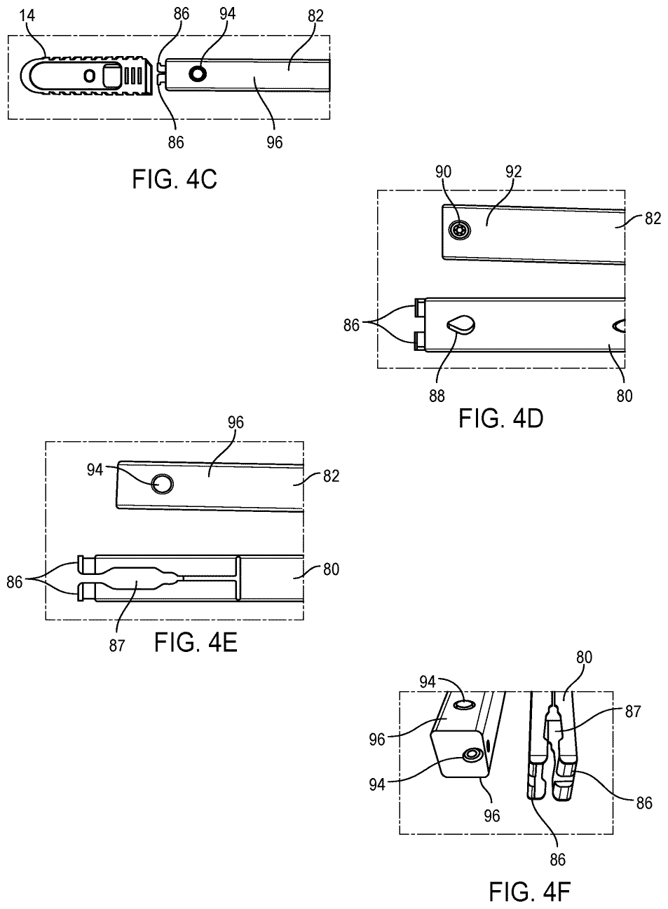

Reference is now made to FIGS. 4A-4F, which show more detailed and assembled views of the implant inserter assembly 18 components.

The inserter tube 80 includes locking fingers, or tips, 86, a flex cutout 87 extending between the locking fingers 86, and a teardrop-shaped aperture 88 at its bifurcated distal end. The locking fingers 86 are configured to releasably engage the undercuts 41 of the posterior surface 36 of interbody implant 14. The release tube 82 includes dimples, or circular indentations, 90 on a first pair of opposed surfaces 92 at its distal end, and interior pins 94 extending inwardly from a second pair of opposed surfaces 96 at its distal end.

The release tube 82 is configured to slide over the inserter tube 80, as shown in FIGS. 4A and 4B, and secured thereto as further described below. When the release tube 82 is slid over the inserter tube 80, the dimples 90 on the release tube 82 interface with the teardrop-shaped aperture 88 on the inserter tube 80 and thereby force the locking fingers 86 of the inserter tube 80 to collapse and converge towards each other, which causes the locking fingers 86 to disengage and release from the interbody implant 14 (i.e., from the undercuts 41 of posterior surface 36, with which the locking fingers 86 engage). This mechanism is further discussed below.

The interior pins 94 on the release tube 82 interface with the inserter flex cutout 87 on the inserter tube 80 to allow the locking fingers 86 of the inserter tube 80 to collapse and to force the locking fingers 86 of the inserter tube 80 open and to interface with the interbody implant 14 when in the locked position.

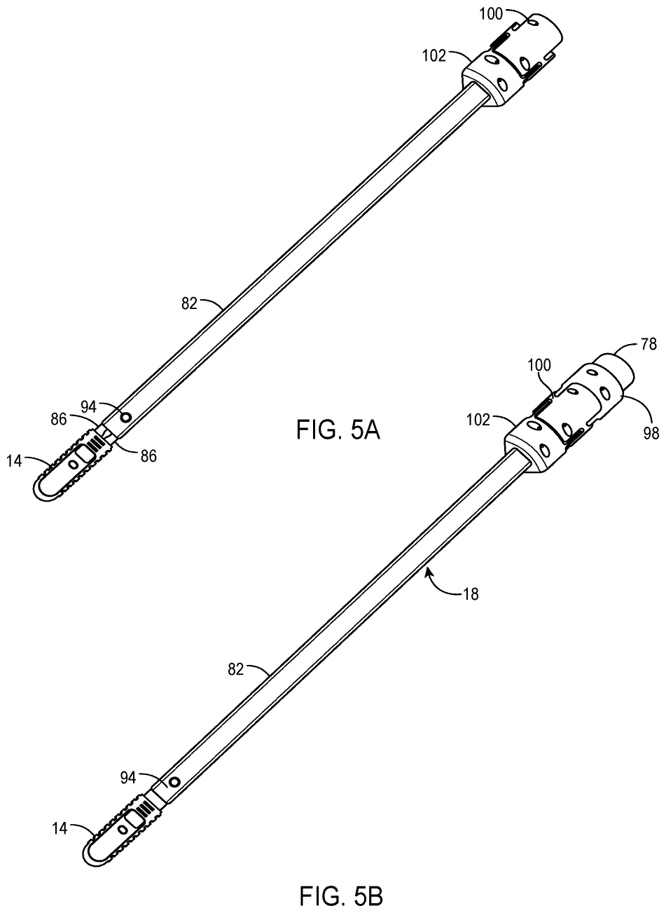

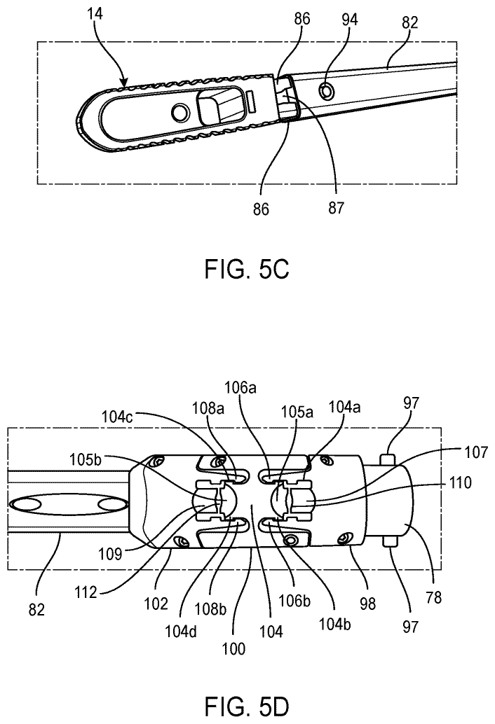

FIG. 5A shows the release tube 82 slid over and secured to the inserter tube 80, and FIG. 5B shows the graft cannula lock tube 78 as inserted into the inserter tube 80 within the release tube 82. The graft cannula lock tube 78 provides a smooth conduit for the bone graft to pass through and keeps the locking fingers 86 of the inserter tube 80 open (i.e., separated) in the locked position and securely locked with the interbody implant 14 (see FIG. 5C). The graft cannula lock tube 78 has two opposed tabs, or protrusions, 97 on its proximal end, the purpose for which is discussed below.

There are two relative positions between the inserter tube 80 and the release tube 82. In the first position (locked) interior pins 94 in the release tube 82 engage the inserter flex cutout 87 in the inserter tube 80 to prevent inserter tip collapse (and implant release) while the dimples 90 on the release tube are in a clearance pocket (i.e., the teardrop-shaped apertures 88) in the inserter tube 80. In the second position (release) the interior pins 94 are in a wider clearance area of the slot/cutout 87 and do not prevent collapse of the tips while the dimples 90 on the release tube 82 press on the tips of the inserter tube 80 actively compressing them together and disengaging from the implant. Forward motion of the release tube 82 also engages the posterior surface 36 of the interbody implant 14 to assist with its release.

As shown in FIGS. 5A, 5B and 5D, connectors 98, 100 and 102 are formed on the respective proximal ends of the graft cannula lock tube 78, the inserter tube 80, and the release tube 82. The inserter tube connector 100 includes a star-shaped latch, or locking member, 104 with proximal arms 104a, 104b and distal arms 104c, 104d, a first proximally extending ramp 105a and a first distally extending ramp 105b. The graft cannula lock tube connector 98 includes distally extending prongs 106a, 106b and a second distally extending ramp 107. The release tube connector 102 includes proximally extending prongs 108a, 108b and a second proximally extending ramp 109, the purposes for which are discussed below.

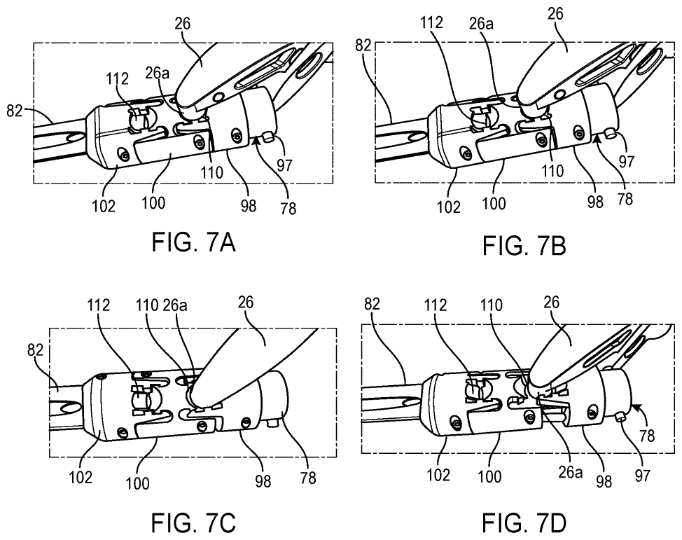

When the release tube 82 is slid over the inserter tube 80, the inserter tube connector 100 engages the release tube connector 102, whereby the proximally extending prongs 108a, 108b receive between them and snap over the distal arms 104c, 104d of the inserter tube connector locking member 104 and secure the release tube 82 to the inserter tube 80 (see FIGS. 4A, 4B, 5A and 5B). Once engaged to each other, the inserter tube connector 100 and the release tube connector 102 define a first enclosed space 110 on each side thereof and between them that contain s first and second distally extending ramps 105b, 109 (see FIGS. 5D, and 7A-7D). Likewise, when the graft cannula lock tube 78 is inserted into the inserter tube 80, the graft cannula lock tube connector 98 engages the inserter tube connector 100, whereby the distally extending prongs 106a, 106b receive between them and snap over the proximal arms 104a, 104b of the inserter tube connector locking member 104 and secure the graft cannula lock tube 78 to the inserter tube 80 and, by extension, the release tube 82 (see FIG. 5B and 5D). Once engaged to each other, the inserter tube connector 100 and the graft cannula lock tube connector 98 define a second enclosed space 112 on each side thereof and between them that contains the first and second proximally extending ramps 105a, 107 (see FIGS. 5D, and 7A-7D). In this way, the connectors 98, 100 and 102 cooperate to keep the graft cannula lock tube 78, the inserter tube 80, and the release tube 82 secured together in a locked position until released with the disassembly pliers 26.

As shown in FIG. 1, the disassembly pliers 26 includes inwardly extending tips 26a, 26b and a compressible handle 27. The inwardly extending tips 26a, 26b are configured to insertably engage the first enclosed spaces 110 (i.e., on both sides of the interconnected connectors 98 and 100) as shown in FIGS. 7A-7D, and, similarly, to later insertably engage the second enclosed spaces 112 (i.e., on both sides of the interconnected connectors 100 and 102), and further discussed below.

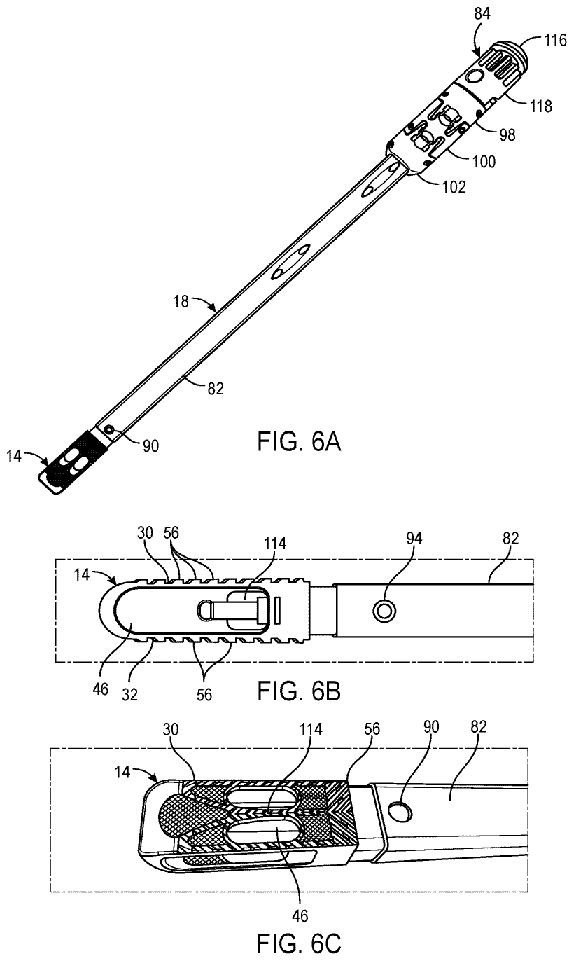

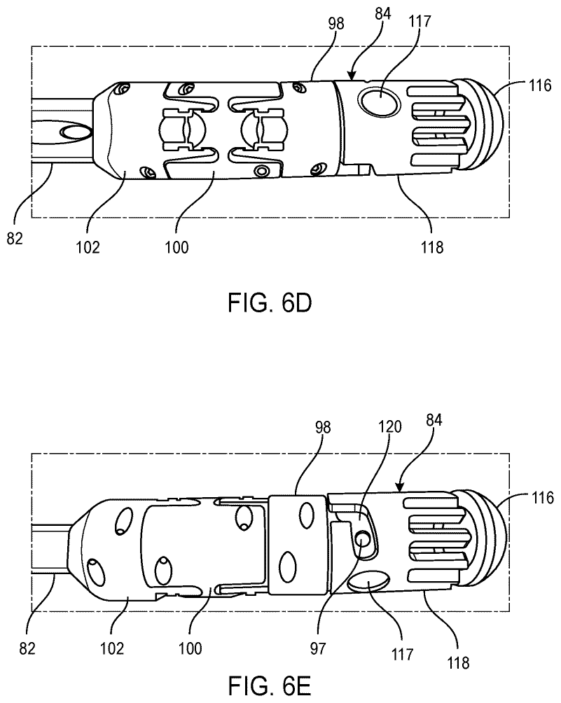

After the graft cannula lock tube 78, the inserter tube 80, and the release tube 82 are secured to each other, the impactor 84 is inserted into the graft cannula lock tube 78 and secured therein, as further explained below and shown in FIGS. 6A-6E. The impactor 84 includes a shaft 114 and a strike cap 116 and rotatable impactor lock sleeve 118 at its distal end. The impactor lock sleeve 118 includes two L-shaped slots 120 that are configured to receive and rotatably engage the two tabs 97 on the connector 98 of the graft cannula lock tube 78 to secure the impactor 84 to/within the graft cannula lock tube 78, as shown in FIG. 6E.

As shown in FIGS. 6B and 6C, the impactor shaft 114 connects the impactor strike cap 116 to the central strut 46 of the interbody implant 14, thereby transmitting the impact force directly to the interbody implant 14 instead of through the inserter tubes 78, 80, 82. The impactor 84 is also self-centering, and thereby avoids damage to the interface between the inserter tube 82 (and other components of the implant inserter assembly 18) and the interbody implant 14.

FIGS. 7A-D show the locking and release mechanism of the telescoping tubes (i.e., the graft cannula lock tube 78, the inserter tube 80, and the release tube 82), and the steps for separating same. The round, inwardly extending tips 26a, 26b of the disassembly pliers 26 are first aligned with the first enclosed spaces 110 on each side of interconnected connectors 98 and 100, as shown in FIG. 7A (see also FIG. 5D). Next, the handle 27 is compressed a first distance, to force the tips 26a, 26b into the respective first enclosed spaces 110, as shown in FIG. 7B. This action forces the distally extending prongs 106a, 106b of the graft cannula lock tube connector 98 to splay apart and release the proximal arms 104a, 104b of the locking member 104. With the proximal arms 104a, 104b released, the continued squeezing action of the pliers 26 (i.e., compressing the handle 27 a second distance) forces the tips 26a, 26b into the first and second proximally extending ramps 105a, 107, which depresses these ramps and thereby disengages the connectors 98 and 100 from each other, as shown in FIG. 7C, such that the connectors 98 and 100, and the graft cannula lock tube 78 and inserter tube 80, separate from each other, as shown in FIG. 7D.

### Graft Funnel and Plunger

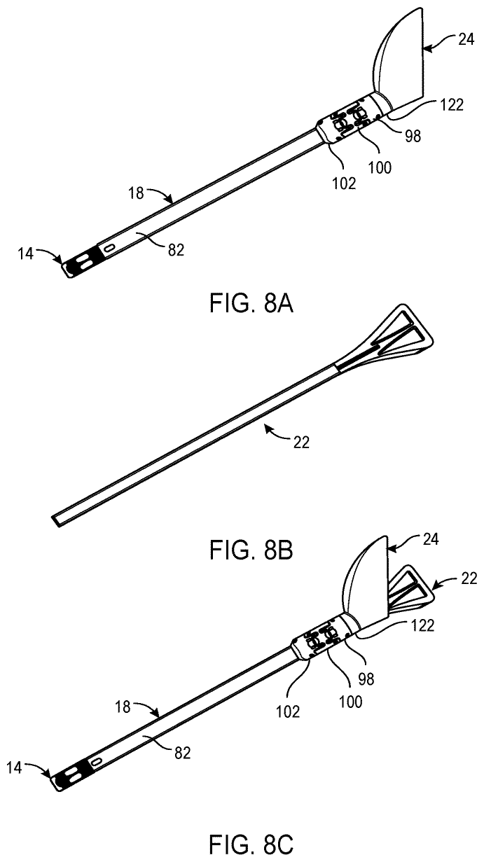

FIGS. 8A-8C shows the graft plunger 22 and graft funnel 24 and of the system 10, as used with the implant inserter assembly 18. After placement of the interbody implant 14 within the patient's disc space, the impactor 84 is disengaged and removed from the graft cannula lock tube 78, and the graft funnel 24 is then secured to the distal end of the graft cannula lock tube 78. In an embodiment, the graft funnel 24 includes a collar 122 having two L-shaped slots 123 (see FIG. 20) formed therein that are similar to the two L-shaped slots 120 of the impactor 84 and that receive and rotatably engage the two tabs 97 of the graft cannula lock tube connector 98 to removably secure the graft funnel 24 to the graft cannula lock tube 78, as shown in FIG. 8A. The graft funnel 24 is a “no-spill” funnel that eliminates or minimizes the loss of bone graft.

The graft funnel 24 is designed to receive and store bone graft when the attached implant inserter assembly 18 is positioned at an angle in the range of approximately 10°-40°, which is typical for PLIF and TLIF surgical procedures. Further, the graft funnel 24 is specially designed to reduce the potential for graft material spilling. The shape of the graft funnel 24 allows for maximal graft security in both the PLIF direction and oblique TLIF direction. Once bone graft is deposited into the graft funnel 24, the graft plunger 22 is inserted (see FIGS. 8B and 8C) through the opening of the graft funnel 24, and is used to advance bone graft from the graft funnel 24 through the graft cannula lock tube 78, through the interbody implant 14 and into the disc space, as also discussed below.

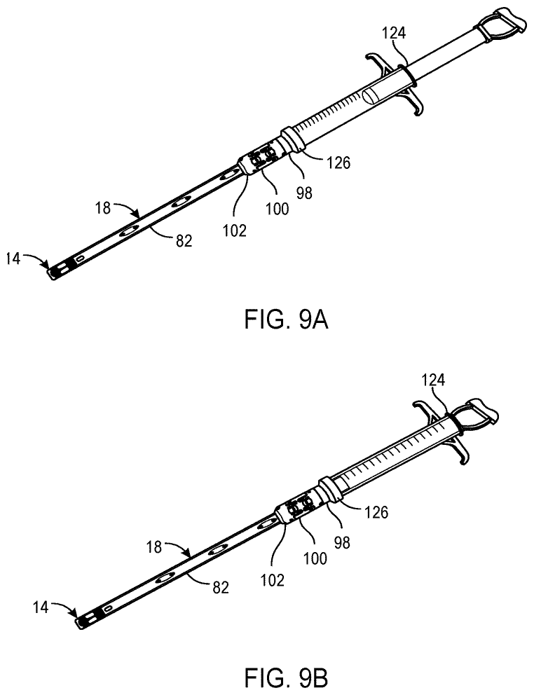

FIGS. 9A and 9B show an alternate embodiment, in which a syringe 124 containing bone graft is attached to the graft cannula lock tube 78 using a syringe adaptor 126 that is configured to removably engage the graft cannula lock tube connector 98. The bone graft is advanced into the graft cannula lock tube 78 at which point the syringe and adaptor 124, 126 are removed, and the graft plunger 22 (not shown) is used to advance the bone graft from the graft cannula lock tube 78, through the interbody implant 14 and into the disc space, as further discussed below.

The syringe adaptor 126 is modular and can be designed to enable the attachment of any graft syringe 124 to the inserter assembly 18. The syringe adaptor is not integral to the syringe 124. In an embodiment, the syringe 124 is specifically configured to engage the inserter assembly 18.

### Cover Plate Inserter

FIG. 10A shows the cover plate inserter 20, and how it inserts into the implant inserter assembly 18. After the bone graft has been moved through the graft cannula lock tube 78 and interbody implant 14 and into the disc space, the graft cannula lock tube connector 98 is disengaged from the inserter tube connector 100 (i.e., using the disassembly pliers 26, as shown in FIGS. 7A-7D and discussed above). The graft cannula lock tube 78 is then removed from the inserter tube 80, whereupon the cover plate inserter 20, with the cover plate 16 attached, is inserted into the inserter tube 80, to engage and attach the cover plate 16 to the posterior opening 42 of the interbody implant 14. The cover plate 16 may be provided in the tray 12 as pre-assembled with the cover plate inserter 20 in its unlocked position. The cover plate 16 is retained on a cover plate retainer 128 at a distal end of the cover plate inserter 20. The cover plate inserter 20 further includes a draw rod tube 130, a hexalobe rod 132 rotatably and concentrically arranged within the draw rod tube 130, and an outer cover plate inserter tube 134. The unlocked and locked positions of the cover plate 16 are shown in FIGS. 10B and 10C, respectively. Engagement of the cover plate locking mechanism is independent of the cover plate's release from the cover plate inserter 20.

FIGS. 11A and 11C show the cover plate inserter 20 as inserted into the inserter tube 80 of the implant inserter assembly 18 in a two-step operation, while FIGS. 11B and 11D show details of the interconnected interbody implant 14 and cover plate 16 in such positions. [FIGS. 11A and 11B show the locked position of the cover plate inserter 20 and interbody implant 14, respectively, and FIGS. 11C and 11D show the unlocked position of the cover plate inserter 20 and interbody implant 14, respectively. In FIG. 11A, the arms 66 on the cover plate 16 are in a locked position. The hexalobe rod 132 is rotated from an unlocked position (shown in FIG. 11C) to a locked position (shown in FIG. 11A), and may include indicia (i.e., an arrow) to identify the rotation direction. More particularly, the hexalobe rod 132 is rotated so that the wings 133a, 133b align with the fingers 136a, 136b on the cover plate inserter outer tube 134, which places the cover plate 16 in the locked position, as shown in 11A and 11C. This rotation is about 30° and rotates the cam 60 of the cover plate 16 to its locked position, which splays the cover plate's arms 66 apart so that they engage with the side cutouts 43a, 43b of the interbody implant 14 (see also FIGS. 10B and 10C). When the cam 60 is rotated to its locked position, it is rotated past the vertical ledges 72 of the arms 66 that keeps the cam 60 from unlocking. In this position the cover plate 16 is engaged with the interbody implant 14 and cannot be removed therefrom. To detach the cover plate 16 from the cover plate inserter 20 (which constitutes a second step), the draw rod tube 130 is rotated counterclockwise until it fully unthreads from the cover plate 16. The draw rod tube 130 and hexalobe rod 132 are then free to travel axially enough to disengage from the cover plate 16 but are retained from separating from the cover plate inserter tube 134 by its fingers 136a, 136b, which are L-shaped and retain the draw rod tube 130 and hexalobe rod 132 in place with respect to the cover plate inserter tube 134. The hexalobe rod 132's locking motion is a counterclockwise rotation so that the cover plate 16 is not inadvertently unlocked while unthreading the draw rod tube 130. Once the cover plate 16 is detached from the cover plate inserter 20, the cover plate inserter 20 is removed from the inserter tube 80 of the implant inserter assembly 18.

The tubes/cannulas of the implant inserter assembly 18 extend past the locking features of the cover plate 16 to ensure that such features are clear of bone graft after grafting, which would interfere with and possible prevent locking the cover plate in place on the interbody implant 14.

### Interbody Implant-Inserter Tube Interface

FIGS. 12A-12D show the release of the interbody implant 14 from the inserter tube 80, which utilizes the same latch mechanism as described above and shown in FIGS. 7A-7D, and as described below. The round, inwardly extending tips 26a, 26b of the disassembly pliers 26 are first aligned with the second enclosed spaces 112 on each side of interconnected connectors 100 and 102, as shown in FIGS. 12A and 12C (see also FIG. 5D). Next, the handle 27 is compressed a third distance, forcing the tips 26a, 26b into the respective second enclosed spaces 112. This action forces the proximally extending prongs 108a, 108b of the release tube 82 to splay apart and release the distal arms 104c, 104d of the locking member 104. With the distal arms 104c, 104d released, the continued squeezing action of the pliers 26 (i.e., compressing the handle 27 a fourth distance) forces the tips 26a, 26b into the first and second distally extending ramps 105b, 109, which depresses these ramps and thereby disengages the connectors 100 and 102 from each other, as shown in FIGS. 12B and 12D, such that the connectors 100 and 102, and the inserter tube 80 and release tube 82, separate from each other. This action also causes the dimples 90 in the release tube 82 to compress the locking fingers 86 of the inserter tube 80 together (i.e., via the teardrop-shaped aperture 88 on the inserter tube 80), which causes the locking fingers 86 to disengage and release from the interbody implant 14 (i.e., from the undercuts 41 of posterior surface 36, with which the locking fingers 86 engage), as shown in FIG. 13.

With further reference to FIG. 13, the interface between the interbody implant 14 and the inserter tube 80 is a critical feature in that it must be very robust but also must be very low profile so as to maximize the size of the graft cannula lock tube 78. The locking fingers 86 of the inserter tube 80 are configured to releasably engage the undercuts 41 of the interbody implant 14 in four locations along its posterior surface 36, and thereby act as hooks. The strength of these locking fingers/hooks 86 is enhanced by the material (i.e., stainless steel) around the comer of the inserter tube 80. This material acts as a strut to significantly enhance the stiffness and strength in bending of the fingers/hooks. The central strut 46 in the interbody implant 14 improves the strength in compressing top surface 30 toward bottom surface 32.

The system 10 is advantageous in that it combines multiple steps into a single pass technique, thereby allowing the surgeon to reduce the risk and amount of nerve trauma in the patient. The system 10 is also designed so that the interbody implant 14 can be removed or revised with readily available tools at any stage of the insertion process, as described below. This feature is a commonly overlooked part of implant insertion surgery, where surgeons are left on their own to figure out a salvage technique.

### Auxiliary Features and Instruments

The system 10 includes further features and instrumentation to facilitate repositioning or removal of the interbody implant 14 at any step of the procedure. FIGS. 14A and 14B show an inserter slap hammer adaptor 138 that is configured to connect to the release tube 82 when the interbody implant 14 is attached and locked to the implant inserter assembly 18 with the graft cannula lock tube 78 in place. The inserter slap hammer adaptor 138 includes a cavity 140 that is configured to receive and cradle the connectors 98, 100 and 102 therein, along with the impactor strike cap 116 and impactor lock sleeve 118. The proximal end 142 of the inserter slap hammer adaptor is configured to connect to a slap hammer 144 that is used to impact the interbody implant 14 in the reverse direction of insertion.

FIGS. 15A-15E show a side grip remover/repositioner instrument 146 that is used to remove or reposition the interbody implant 14 once it has been implanted in the disc space. The remover/repositioner instrument 146 includes an internal rod 148, a bifurcated distal end 150 having two prongs 152a, 152b on either side of the internal rod 148, a threaded portion 154 operably engaged with a proximal end of the internal rod 148, a nut member 156 and a knob 158 at a distal end 160 of the remover/repositioner instrument 146. The knob 158 is externally threaded (not shown), and moveably engageable with the threaded portion 154, such that rotation of the knob 158 causes axial movement of the internal rod 148. The bifurcated distal end 150 and internal rod 148 are dimensioned to fit through the inserter tube 80 of the implant inserter assembly 18, such that the remover/repositioner instrument 146 can insertably engage the combined inserter tube 80 and release tube 82 when attached to the interbody implant 14 (see FIG. 15B). When the remover/repositioner instrument 146 is fully inserted into the combined inserter tube 80 and release tube 82, the knob 158 (and proximal end 160) is rotated to threadably engage the threaded portion 154 and nut member 156, which causes the internal rod 148 to advance and splay the prongs 152a, 152b of the bifurcated distal end 150 apart so that they engage with the respective side cutouts 43a, 43b of the interbody implant 14 (see FIGS. 15E and 15F). At this point the remover/repositioner instrument 146 is securely attached to the interbody implant 14 and the slap hammer 144 (not shown, but see above and FIGS. 14A and 14B) may be attached to the combined inserter tube 80 and release tube 82 to remove the interbody implant 14 from the disc space, or reposition the interbody implant 14 in the disc space.

The remover/repositioner instrument 146 must be used without the cover plate 16 attached to the interbody implant 14. In some embodiments, the remover/repositioner instrument 146 may be used without the combined inserter tube 80 and release tube 82 attached to the interbody implant 14. In other words, the remover repositioner 148 can be attached to the posterior surface 36 (i.e., proximal end) of the interbody implant 14 directly, and operate using the same method for expansion of the bifurcated distal end 150 as described above.

FIG. 16A-16C show a hooked remover/repositioner instrument 162 that provides an alternate means with which to remove or reposition the interbody implant 14 once it has been implanted in the disc space (i.e., once the inserter tube 80 and release tube 82, have been removed from the disc space). The hooked remover/repositioner instrument 162 includes a shaft 164 with a proximal end 166 and a distal hook 168. In operation, the distal hook 168 of the hooked remover/repositioner instrument 162 is inserted through the posterior opening 42 of the interbody implant 14 and hooked around one of the opposed side surfaces 34a, 34b, as shown in FIG. 1 6C. The slap hammer 144 (not shown, but see above and FIGS. 14A and 14B) is then be attached to the proximal end 166 to remove the interbody implant 14 from the disc space, or reposition the interbody implant 14 in the disc space.

FIG. 17A-17C show a threaded remover/repositioner instrument 170 that provides another alternate means with which to remove or reposition the interbody implant 14 with the cover plate 16 attached thereto, once the implant 14 has been implanted in the disc space (i.e., once the inserter tube 80 and release tube 82, have been removed from the disc space). The threaded remover/repositioner instrument 170 can be used with or without the inserter tube 80 connected to the interbody implant 14. The threaded remover/repositioner instrument 170 includes a shaft 172 with a proximal end 174 and a threaded distal tip 176 that is configured to threadably engage the threaded opening 62 of the cover plate 16. In operation, the threaded distal tip 176 of the threaded remover/repositioner instrument 170 is inserted into the threaded opening 62 of the cover plate 16 and threadably secured thereto (see FIGS. 17B and 1 7C). The slap hammer 144 (not shown, but see above and FIGS. 14A and 14B) is then be attached to the proximal end 174 to remove the interbody implant 14 and cover plate 16 from the disc space, or reposition the interbody implant 14 in the disc space.

The serially-arranged cannulas/tubes of the implant inserter assembly 18 enables the surgeon to robustly attach the tubes to the implant interface, both having the same cross-sectional shape, such as rectangular (as shown herein), oval or other possible shapes. This configuration eliminates creating a “wasp-waist” bottleneck between the cannula and the implant. Retaining a constant cross-sectional area through the cannula/implant junction and through the exit zones of the implant, and maintaining a robust connection between the cannula and implant, improves the delivery of bone graft and patient outcomes.

The system 10 further provides a secure and coordinated locking and unlocking mechanism between the cannulas/tubes of the implant inserter assembly 18 and the interbody implant 14 at opposite ends of the implant inserter assembly 18. The structure of the connectors 98, 100, 102 of the of the implant inserter assembly 18 (including the locking member/latch 104, prongs 106a, band 108a, b, and ramps 105a, b, 107 and 109) and disassembly pliers 26, as described above, enables a single motion stepwise release including sequential unlocking and displacing steps that prevent the uncontrolled release of the lock and subsequent unintended motion of the cannulas/tubes. Further, the release of the graft cannula lock tube 78 is advantageously completed without interfering with the attachment between the interbody implant 14 and cannulas (i.e., the inserter tube 80 and release tube 82). The second release of the “surgeon side” (i.e., proximal) cannula attachment with the pliers 26 simultaneously forces the tabs depress at the cannula/implant interface to release the implant, as described above and shown in FIGS. 7A-D and 12A-13.

A further advantageous and novel aspect of the system 10 is that it combines implant insertion and graft delivery with an interbody implant, and allows for a “touch-free” device that does not require pre-packing the disc space or implant.

In an embodiment, the system 10 facilitates anti-embolism air release. Portals 178 are formed in all three tubes/cannulas of the implant inserter assembly 18 (see FIGS. 4B, 10A, 11C, 12A and 12B) and constitute a path to release air trapped within the implant inserter assembly 18. The portals prevent the transmission of air into the disc space that could be forced into the venous communication and bleeding bone within the disc space. If air is forced into these bleeding chance, it can produce an air embolism.

### Surgical Methods for Implantation

Exemplary surgical methods for using the system 10 will now be described.

The patient is placed in a prone position for posterior access to the spine. The patient's target intervertebral disc space is exposed for the desired level in the usual fashion.

Standard disc spreaders and/or shavers are used to distract the disc space. Adequate distraction is one of the preconditions for the primary stability of the interbody implant; however, it is critical to ensure that the segment is not over- distracted to avoid damage of ligaments and/or endplates.

A combination of curettes, rasps, osteotomes, and other appropriate surgical instruments is used to remove the disc material and cartilage from the vertebral endplates on both sides of the disc space. Appropriate removal of the cartilaginous layers of the endplates is important for the vascularization of the bone graft. The endplates are cleaned carefully and integrity of the underlying bony endplate is maintained, as damage of the endplate can lead to implant subsidence.

The surgeon then selects the interbody implant. Trial spacers are available to provide guidance prior to implant selection. The height of the trial spacer matches the height of the corresponding implant. Trials may be used to determine appropriate implant height and length. The implant selection process may include the following steps in an embodiment:
a. Attach a I-handle to a Hudson connect end of the desired trial. Start with the smallest trial and use subsequent trial heights until the desired fit is achieved.
b. Use a mallet to impact the trial into the disc space and the desired position.
c. Use fluoroscopy to confirm the trial location. Determine the implant length using the fluoroscopy image and the length to the trial cutouts and back of trial head.
d. Consider applying contralateral, provisional pedicle screw fixation to maintain disc space distraction to facilitate end plate preparation.
e. Remove the T-handle from the trial shaft and replace with the slap hammer to remove the trial from the disc space.

The interbody implant is available m a wide variety of sizes to ensure appropriate sizing of the implanted components. Correct size selection is critical to the surgical outcome. An under- or oversized implant can lead to premature failure thereof.

The appropriate pre-packed sterile interbody implant is selected, based on trialing in the previous step. The exterior packaging (e.g., box) is opened, and the sterile implant/inserter kit is then delivered to the sterile field in the normal manner for a double Tyvek pouch.

Referring again to the drawings, and FIG. 18 specifically, the interbody implant 14 comes preassembled with the implant inserter assembly 18, including the impactor 84. A mallet (not shown) is used to tap on the impactor 84 to advance the implant 14 into the disc space DS (i.e., between adjacent vertebrae VI, V2), as shown by arrow M. The position of the implant 14 within the disc space DS is confirmed with fluoroscopy.

As discussed above, the interbody implant 14 has teeth 56 on its top and bottom surfaces 30, 32 to maximize primary stability (see FIGS. 2A, 6C and 13). The soft tissue and dura must be adequately retracted when inserting the implant 14 to avoid damage from contact with the implant' s surfaces, in particular the teeth 56 thereon. Adequate implant positioning is critical; an improperly placed implant can adversely affect device performance and/or surgical outcome.

With reference to FIGS. 19A and 19B, the impactor 84 is carefully removed from the inserter assembly 18 by first rotating the impactor lock sleeve 118 (shown by arrow A), thereby disengaging the L-shaped slots 120 of the impactor 84 from the tabs 97 on the graft cannula lock tube 78 (as shown in FIG. 6E), and then pulling the impactor 84 out of and away from the inserter assembly 18 (shown by arrow B). The disassembly pliers 26 may be used to unlock the lock sleeve 118 and remove the impactor 84. These pliers may be rotated for optimal positioning. Removal of the impactor 84 exposes the open-ended the graft cannula lock tube 78 of the inserter assembly 18, as shown in FIG. 19B.

As shown in FIG. 20, the graft funnel 24 is then attached to the inserter assembly 18 (i.e., by cooperation between the L-shaped slots 123 formed on funnel collar 122 and the two tabs 97 of the graft cannula lock tube 78). The bone graft may then be deposited into the graft funnel 24. In various embodiments, the bone graft may include autogenous and/or allogenic bone graft comprised of cancellous and/or corticocancellous bone graft.

Once the bone graft has been deposited into the graft funnel 24, the graft plunger 22 is inserted (see FIGS. 8B and 8C) through the opening of the graft funnel 24. In this fashion, the surgeon advances bone graft from the graft funnel 24 through the graft cannula lock tube 78, through the interbody implant 14 and into the disc space DS. These steps are further described below.

The graft funnel 24 is used as a reservoir for the bone graft. In an embodiment, 1-2 ml of bone graft is deposited/inserted into the graft cannula lock tube 78 of the inserter assembly 18 at a time, and the graft plunger 22 is used to advance the bone graft through the graft cannula lock tube 78 and into the open center 44 of the interbody implant 14 (i.e., cage). More flowable graft materials can be applied into the graft cannula lock tube 78 as a single aliquot. The graft plunger 22 is then completely depressed, as shown by arrow C in FIG. 21, to completely fill the open center 44 of the implant 14 and disc space DS. This process is continued until the disc space DS and implant 14 are full of bone graft, i.e., until no more bone graft can be pushed into the disc space. The disc space DS is filled when flow resistance is felt and/or if the graft plunger 22 no longer bottoms out in the graft funnel 24 Additional bone graft should not be forced into the disc space at this point. At this point, the graft plunger 22 and graft funnel 24 are removed from the graft cannula lock tube 78/inserter assembly 18. The disassembly pliers 26 may then be used to remove the graft cannula lock tube 78 from the inserter tube 80 and remaining inserter assembly 18, as shown by arrow D in FIG. 22A. FIG. 22C shows the remaining inserter assembly 18 (i.e., the combined inserter tube 80 and release tube 82) after removal of the graft cannula lock tube 78.

FIGS. 23A-23D show the steps of using the cover plate inserter 20 to insert and attach the cover plate 16 to the posterior surface 36 (i.e., proximal end) of the interbody implant 14. The cover plate inserter 20 with the cover plate 16 attached is inserted into the inserter tube 80 of the implant inserter assembly 18, as shown by arrow E in FIG. 23A. The surgeon knows when the cover plate 16 has been placed in the proper position when the cover plate inserter 20 bottoms out on the inserter tube 80. The surgeon then rotates the smaller, most proximal knob (i.e., the cover plate inserter's hexalobe rod 132) of the cover plate inserter 20 about ¼ tum counterclockwise (as shown by arrow F in FIG. 23B) to lock the cover plate 16 in place on the posterior surface 36 (i.e., proximal end) of the interbody implant 14. The surgeon then confirms that the cover plate 16 is secured to the implant 14 by gently pulling on the cover plate inserter 20. The surgeon then rotates the larger, most distal knob (draw rod tube 130) on the cover plate inserter 20 counterclockwise several turns (as shown by arrow G in FIG. 23C) to unthread and disengage the cover plate inserter 20 from the cover plate 16. The cover plate inserter 20 is then removed from the inserter tube 80 of the implant inserter assembly 18, as shown by arrow H in FIG. 23D.

It should be noted that the cover plate 16 is optional, and its use is not required for the spinal procedure. The cover plate may 16 be used is at the discretion of the surgeon.

FIGS. 24A-24B show the steps for removing the release tube 82 and release the inserter 80 from the interbody implant 14. The disassembly pliers 26 are first used to advance the release tube 82, as shown by arrow I in FIG. 24A, and thereby release the inserter tube 80 from the implant 14. The inserter tube 80 is then removed from the disc space DS, followed by the release tube 82, as shown by the arrow J in FIG. 24B. FIG. 24C shows the implant 14 placed in the disc space after all components of the inserter system 10 have been removed therefrom. After these steps, the surgeon should then observe the area around the annulotomy to confirm that bone graft has not overflowed or was spilled when removing the inserter tube 80. The cover plate 16 is used in the case of soft flowable bone graft.

After the foregoing steps, radiographic verification may be used to verify the final implant placement. Such verification incudes anterior/posterior and lateral fluoroscopy images of the disc space and implant.

The interbody implant 14 is designed to withstand full load-bearing until bony union of the spinal segment(s)/vertebrae normally occurs. To ensure load-bearing capability, supplemental fixation is required for use with these implants.

### Implant Repositioning and Removal

The interbody implant 14 is intended for permanent implantation and is not intended to be removed in case of a good outcome. However, adverse events might warrant removal of the implant, or require it to be repositioned. There are four options to reposition or remove the implant, as discussed below.

a. Using an Inserter Slap Hammer Adaptor

A slap hammer 144 and inserter slap hammer adaptor 138 are described above and shown in FIGS. 14A-14B. As shown in FIG. 25, the inserter slap hammer adaptor 138 is fit around the implant release tube 82 (i.e., the connector 102 thereof) and ensure the implant release tube connector 102 is fully seated in the cavity 140 on the inserter slap hammer adaptor 138. The slap hammer 144 is used to apply a removal force to the inserter assembly 18 and interbody implant 14, as shown by arrow K. Care is to be used to protect the patient's sensitive anatomy during slap hammering, as the implant 14 may move unexpectedly during slap hammering.

The inserter slap hammer adaptor 138 should not be used if the graft cannula lock tube 78 has been removed from the inserter tube 80. The graft cannula lock tube 78 must be fully engaged to ensure the connection between the inserter tube 80 and interbody implant 14. is secure. The graft cannula lock tube is used to ensure proper fixation of the inserter tube 80 to the implant 14 before repositioning or removal of the implant 14 from the disc space.

b. Using a Side Grip Remover/Repositioner Instrument

A side grip remover/repositioner instrument 146 is described above and shown in FIGS. 15A-15E. The cover plate 16 (if attached/in place on the interbody implant 14) is first unlocked and removed from the implant 14. The side grip remover/repositioner instrument 146 is then used to engage the implant 14 by rotating the knob 158 until locked. The slap hammer 144 is then connected to the combined inserter tube 80 and release tube 82 to remove the interbody implant 14 from the disc space, or reposition the interbody implant 14 in the disc space, as shown by arrow L in FIG. 26A. In an alternate embodiment, the side grip remover/repositioner instrument 146 is employed without the combined inserter tube 80 and release tube 82 attached to the interbody implant 14, as shown by arrow N in FIG. 26B.

c. Using a Hooked Remover/Repositioner Instrument

A hooked remover/repositioner instrument 162 is described above and shown in FIGS. 16A-16C. First, the implant inserter assembly 18 (i.e., the inserter tube 80 and release tube 82) are removed from the disc space, as shown by arrow P in FIG. 27A. The distal hook 168 is then used to engage the interbody implant 14 via its posterior opening 42. The slap hammer 144 is then connected to the hooked remover/repositioner instrument 162 to remove the interbody implant 14 from the disc space, or reposition the interbody implant 14 in the disc space, as shown by arrow Q in FIG. 27B.

d. Using a Threaded Remover/Repositioner Instrument

A threaded (i.e., cover plate grip) remover/repositioner instrument 170 is described above and shown in FIGS. 1 7A-1 7C. The t threaded remover/repositioner is threaded into the back of the cover plate 16, which must be secured to the implant 14 in this case. The slap hammer 144 is then connected to the combined inserter tube 80 and release tube 82 to remove the interbody implant 14 from the disc space, or reposition the interbody implant 14 in the disc space, as shown by arrow R in FIG. 28A. In an alternate embodiment, the threaded remover/repositioner instrument 170 is employed without the combined inserter tube 80 and release tube 82 attached to the interbody implant 14, as shown by arrow Sin FIG. 28B.

After the interbody implant 14 is placed, the extradural space and foramina are probed to ensure adequate decompression of the neural elements. Final imaging in the AP and lateral planes is recommended.

To facilitate the necessary immobilization of the grafted disc space, segmental fixation may be applied using standard techniques. The wound is then irrigated and closed per surgeon preference.

Components of the embodiments of the invention are formed from materials having properties that enable the components to perform their respective functions. Such materials are biocompatible, and include, for example, stainless steels. The disposable instruments discussed here are made from stainless steel and medical grade polycarbonate.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other examples are also within the claims.

In general, any combination of disclosed features, components and methods described herein is possible. Steps of a method can be performed in any order that is physically possible.

All cited patents, patent publications and non-patent references are incorporated by reference herein in their entireties.

Although embodiments have been disclosed, the invention is not limited thereby.

We claim:

1. A surgical system comprising:
(a) an interbody implant 14, including an open body 28 having top and bottom surfaces 30, 32, opposed side surfaces 34a, 34b, and posterior and anterior surfaces 36, 38, an open center 44 and a central strut 46 extending longitudinally through the open center 44, from the anterior surface towards the posterior surface,
the posterior surface including a posterior opening 42 extending along at least 30% of the posterior surface 36 between the side surfaces 34a, 34b, and the side surfaces 34a, 34b each including a side opening 40a, 40b extending along at least 50% of the side surfaces 34a, 34b,
wherein the side openings 40a, 40b and posterior opening 42 allow for bone graft to flow through the posterior surface 36 of the interbody implant 14 and through the side surfaces 34a, 34b and into a patient's surrounding disc space;
(b) an implant inserter assembly 18, including a graft cannula lock tube 78, an inserter tube 80 configured to telescopically receive the graft cannula lock tube 78 therein, a release tube 82 configured to telescopically receive the inserter tube therein, an impactor 84 configured to insertably engage the graft cannula lock tube 78, inserter tube 80 and release tube 82, and means for releasably interconnecting the graft cannula lock tube 78, inserter tube 80 and release tube 82, wherein the graft cannula lock tube 78, inserter tube 80 and release tube 82 cooperate to form a serial cannula system, and have uniform internal areas to improve transmission of bone graft therethrough and into the posterior opening 42 of the interbody implant 14; and
(c) means for releasably connecting the implant inserter assembly to the interbody implant.

2. The surgical system of claim 1, wherein the means for releasably connecting the implant inserter assembly to the interbody implant includes at least one undercut 41 formed in the posterior surface 36 of the interbody implant 14, and locking fingers 86 formed on a distal end of the inserter tube 80 and configured to releasably engage the undercuts 41.

3. The surgical system of claim 1, wherein the means for releasably connecting the implant inserter assembly to the interbody implant further includes a flex cutout 87 extending between the locking fingers 86, a teardrop-shaped aperture 88 at a distal end of the inserter tube 80, dimples 90 formed on a first pair of opposed surfaces 92 of the release tube 82 and interior pins 94 extending inwardly from a second pair of opposed surfaces 96 of the release tube 82; and
wherein when the release tube 82 receives the inserter tube 80 therein, the dimples 90 interface with the teardrop-shaped aperture 88 and thereby force the locking fingers 86 to collapse and converge towards each other, whereby the locking fingers 86 disengage and release from the undercuts 41 of the interbody implant 14.

4. The surgical system of claim 3, wherein the means for releasably interconnecting the graft cannula lock tube 78, inserter tube 80 and release tube 82 includes a graft cannula lock tube connector 98 formed on a proximal end of the graft cannula lock tube 78 and having distally extending prongs 106a, 106b, an inserter tube connector 100 formed on a proximal end of the inserter tube 80 and having a star-shaped latch 104 with proximal arms 104a, 104b and distal arms 104c, 104d, and a release tube connector 102 formed on a proximal end of the release tube 82 and having proximally extending prongs 108a, 108b;
wherein the proximally extending prongs 108a, 108b are configured to receive between them and snap over the distal arms 104c, 104d of the inserter tube connector locking member 104 and secure the release tube 82 to the inserter tube 80, whereby the inserter tube connector 100 and the release tube connector 102 define a first enclosed space 110 on each side of the implant inserter assembly 18; and
wherein the distally extending prongs 106a, 106b are configured to receive between them and snap over the proximal arms 104a, 104b of the inserter tube connector locking member 104 and secure the graft cannula lock tube 78 to the inserter tube 80, whereby the inserter tube connector 100 and the graft cannula lock tube connector 98 define a second enclosed space 112 on each side of the implant inserter assembly 18.

5. The surgical system of claim 4, further comprising disassembly pliers 26 having inwardly extending tips 26a, 26b and a compressible handle 27,
wherein the inserter tube connector 100 further includes a first proximally extending ramp 105a and a first distally extending ramp 105b, wherein the graft cannula lock tube connector 98 includes a second distally extending ramp 107, wherein the release tube connector 102 includes a second proximally extending ramp 109,
wherein each of the first enclosed spaces 110 contains the first and second distally extending ramps 105b, 109, and is configured to engage the first tip 26a or the second tip 26b;
wherein each of the second enclosed spaces 112 contains the first and second proximally extending ramps 105a, 107, and is configured to engage the first tip 26a or the second tip 26b; and
wherein the handle 27 is compressible a first distance to force the inwardly extending tips 26a, 26b into the respective first enclosed spaces 110, to force the distally extending prongs 106a, 106b of the graft cannula lock tube connector 98 to splay apart and release the proximal arms 104a, 104b of the locking member 104, and is compressible a second distance to force the inwardly extending tips 26a, 26b into and to depress the first and second proximally extending ramps 105a, 107, whereby the connectors 98 and 100 are disengaged from each other and the graft cannula lock tube 78 and inserter tube 80 separate from each other.

6. The surgical system of claim 5, wherein the handle 27 is compressible a third distance to force the inwardly extending tips 26a, 26b of the disassembly pliers 26 into the respective second enclosed spaces 112 to force the proximally extending prongs 108a, 108b of the release tube 82 to splay apart and release the distal arms 104c, 104d of the locking member 104, and is compressible a fourth distance to force the inwardly extending tips 26a, 26b into and to depress the first and second distally extending ramps 105b, 109, whereby the connectors 100 and 102 are disengaged from each other and the inserter tube 80 and release tube 82 separate from each other; and
wherein the dimples 90 in the release tube 82 compress the locking fingers 86 of the inserter tube 80 together via the teardrop-shaped aperture 88 on the inserter tube 80 to disengage and release the locking fingers 86 from the interbody implant 14, whereby the inserter tube 80 separates from the interbody implant 14.

7. The surgical system 10 of claim 1, further comprising a graft funnel 24 configured to removably engage the distal end of the graft cannula lock tube 78, and a graft plunger 22 configured to insertably engage an opening of the graft funnel 24 and to advance bone graft from the graft funnel 24 through the graft cannula lock tube 78, through the interbody implant 14 and into the disc space.

8. The surgical system 10 of claim 1, further comprising an interbody cover plate 16 configured to removably engage the posterior opening 42 of the interbody implant 14, the cover plate 16 including a cover plate body 58 and a cover plate cam 60.

9. The surgical system 10 of claim 8, further comprising a cover plate inserter 20,
wherein the cover plate body 58 includes a posterior surface 59 defining a threaded opening configured for engagement with the cover plate inserter 20 and two integrally-formed arms 66 extending from the cover plate body 58 on opposed sides of the threaded opening 62 and configured to engage the side surfaces 34a, 34b of the interbody implant and lock the cover plate 16 to the interbody implant 14, and
wherein the cam 60 includes a hexalobe feature 74 for engagement with the cover plate inserter 20.

10. The surgical system 10 of claim 9, wherein the arms each include a flex zone area 68, an outwardly-extending wedge-shaped end 70 and a vertically-extending tab 72 adjacent the wedge-shaped end 70,
wherein the arms 66 are configured to flex outwardly by bending at the flex zone area 68,
wherein the wedge-shaped ends 70 of the arms 66 are configured to insertably engage side cutouts 43a, 43b formed in opposed side surfaces 34a, 34b of the interbody implant 14 and lock the cover plate 16 to the interbody implant 14, and
wherein the tabs 72 are configured to engage with the cam 60.

11. The surgical system 10 of claim 10, wherein the cam 60 is rotatable relative to the cover plate body 58, wherein the cam 60 is configured to engage the arms 66 during rotation and to force the arms 66 to flex outwardly by bending at the flex zone area 68, whereby the locking members 76 of the cam 60 engage with the tabs 72 to prevent reverse rotation and lock the arms 66 in an outward flexed orientation.

12. The surgical system 10 of claim 1, wherein the posterior opening 42 of the interbody implant 14, the graft cannula lock tube 78, the inserter tube 80 and the release tube 82 each have rectangular cross-sectional shapes.

13. A surgical system comprising:
(a) an interbody implant 14, including an open body 28 having top and bottom surfaces 30, 32, opposed side surfaces 34a, 34b, and posterior and anterior surfaces 36, 38, an open center 44 and a central strut 46 extending longitudinally through the open center 44, from the anterior surface towards the posterior surface,
the posterior surface including a posterior opening 42 extending along at least 30% of the posterior surface 36 between the side surfaces 34a, 34b, and the side surfaces 34a, 34b each including a side opening 40a, 40b extending along at least 50% of the side surfaces 34a, 34b,
wherein the side openings 40a, 40b and posterior opening 42 allow for bone graft to flow through the posterior surface 36 of the interbody implant 14 and through the side surfaces 34a, 34b and into a patient's surrounding disc space;
(b) an implant inserter assembly 18, including a graft cannula lock tube 78, an inserter tube 80 configured to telescopically receive the graft cannula lock tube 78 therein, a release tube 82 configured to telescopically receive the inserter tube therein, an impactor 84 configured to insertably engage the graft cannula lock tube 78, inserter tube 80 and release tube 82, and means for releasably interconnecting the graft cannula lock tube 78, inserter tube 80 and release tube 82, wherein the graft cannula lock tube 78, inserter tube 80 and release tube 82 cooperate to form a serial cannula system, and have uniform internal areas to improve transmission of bone graft therethrough and into the posterior opening 42 of the interbody implant 14;
(c) means for releasably connecting the implant inserter assembly to the interbody implant;
(d) an interbody cover plate 16 configured to removably engage the posterior opening 42 of the interbody implant 14, the cover plate 16 including a cover plate body 58 and a cover plate cam 60; and
(e) a cover plate inserter 20, wherein the cover plate body 58 includes a posterior surface 59 defining a threaded opening configured for engagement with the cover plate inserter 20 and two integrally-formed arms 66 extending from the cover plate body 58 on opposed sides of the threaded opening 62 and configured to engage the side surfaces 34a, 34b of the interbody implant 14 and lock the cover plate 16 to the interbody implant 14, and wherein the cam 60 includes a hexalobe feature 74 for engagement with the cover plate inserter 20.

14. The surgical system of claim 13, wherein the means for releasably connecting the implant inserter assembly to the interbody implant includes at least one undercut 41 formed in the posterior surface 36 of the interbody implant 14, and locking fingers 86 formed on a distal end of the inserter tube 80 and configured to releasably engage the undercuts 41.

15. The surgical system of claim 13, wherein the means for releasably connecting the implant inserter assembly to the interbody implant further includes a flex cutout 87 extending between the locking fingers 86, a teardrop-shaped aperture 88 at a distal end of the inserter tube 80, dimples 90 formed on a first pair of opposed surfaces 92 of the release tube 82 and interior pins 94 extending inwardly from a second pair of opposed surfaces 96 of the release tube 82,
wherein when the release tube 82 receives the inserter tube 80 therein, the dimples 90 interface with the teardrop-shaped aperture 88 and thereby force the locking fingers 86 to collapse and converge towards each other, whereby the locking fingers 86 disengage and release from the undercuts 41 of the interbody implant 14.

16. The surgical system of claim 15, further comprising disassembly pliers 26 having inwardly extending tips 26a, 26b and a compressible handle 27,
wherein the inserter tube connector 100 further includes a first proximally extending ramp 105a and a first distally extending ramp 105b, wherein the graft cannula lock tube connector 98 includes a second distally extending ramp 107, wherein the release tube connector 102 includes a second proximally extending ramp 109,
wherein each of the first enclosed spaces 110 contains the first and second distally extending ramps 105b, 109, and is configured to engage the first tip 26a or the second tip 26b;
wherein each of the second enclosed spaces 112 contains the first and second proximally extending ramps 105a, 107, and is configured to engage the first tip 26a or the second tip 26b;
wherein the handle 27 is compressible a first distance to force the inwardly extending tips 26a, 26b into the respective first enclosed spaces 110, to force the distally extending prongs 106a, 106b of the graft cannula lock tube connector 98 to splay apart and release the proximal arms 104a, 104b of the locking member 104, and is compressible a second distance to force the inwardly extending tips 26a, 26b into and to depress the first and second proximally extending ramps 105a, 107, whereby the connectors 98 and 100 are disengaged from each other and the graft cannula lock tube 78 and inserter tube 80 separate from each other.

17. The surgical system of claim 16, wherein the handle 27 is compressible a third distance to force the inwardly extending tips 26a, 26b of the disassembly pliers 26 into the respective second enclosed spaces 112 to force the proximally extending prongs 108a, 108b of the release tube 82 to splay apart and release the distal arms 104c, 104d of the locking member 104, and is compressible a fourth distance to force the inwardly extending tips 26a, 26b into and to depress the first and second distally extending ramps 105b, 109, whereby the connectors 100 and 102 are disengaged from each other such and the inserter tube 80 and release tube 82 separate from each other.

18. The surgical system 10 of claim 13, further comprising a graft funnel 24 configured to removably engage the distal end of the graft cannula lock tube 78, and a graft plunger 22 configured to insertably engage an opening of the graft funnel 24 and to advance bone graft from the graft funnel 24 through the graft cannula lock tube 78, through the interbody implant 14 and into the disc space.

19. A kit for spinal surgery, comprising:
(a) an interbody implant 14, including an open body 28 having top and bottom surfaces 30, 32, opposed side surfaces 34a, 34b, and posterior and anterior surfaces 36, 38, an open center 44 and a central strut 46 extending longitudinally through the open center 44, from the anterior surface towards the posterior surface,
the posterior surface including a posterior opening 42 extending along at least 30% of the posterior surface 36 between the side surfaces 34a, 34b, and the side surfaces 34a, 34b each including a side opening 40a, 40b extending along at least 50% of the side surfaces 34a, 34b,
wherein the side openings 40a, 40b and posterior opening 42 allow for bone graft to flow through the posterior surface 36 of the interbody implant 14 and through the side surfaces 34a, 34b and into a patient's surrounding disc space;
(b) an implant inserter assembly 18 releasably connectable to the interbody implant 14 and including a graft cannula lock tube 78, an inserter tube 80 configured to telescopically receive the graft cannula lock tube 78 therein, a release tube 82 configured to telescopically receive the inserter tube therein, an impactor 84 configured to insertably engage the graft cannula lock tube 78, inserter tube 80 and release tube 82, wherein the graft cannula lock tube 78, inserter tube 80 and release tube 82 cooperate to form a serial cannula system, and have uniform internal areas to improve transmission of bone graft therethrough and into the posterior opening 42 of the interbody implant 14;
(c) an interbody cover plate 16 configured to removably engage the posterior opening 42 of the interbody implant 14;
(d) a cover plate inserter 20 configured for engagement with the cover plate 16;
(e) disassembly pliers 26 configured to disconnect the graft cannula lock tube 78, inserter tube 80 and release tube 82 from one another;
(f) a graft funnel 24 configured to removably engage the distal end of the graft cannula lock tube 78;
(g) a graft plunger 22 configured to insertably engage an opening of the graft funnel 24 and to advance bone graft from the graft funnel 24 through the graft cannula lock tube 78, through the interbody implant 14 and into the disc space; and
(h) means for removing or repositioning the interbody implant 14.

20. The kit of claim 19, wherein the means for removing or repositioning the interbody implant is selected from a group consisting of a slap hammer, an inserter slap hammer adaptor, a side grip remover/repositioner instrument, a hooked remover/repositioner instrument and a threaded remover/repositioner instrument.
